(19) United States
(12) Patent Application Publication
(10) Pub. No.: US 2014/0171918 A1
(43) Pub. Date: Jun. 19, 2014

(54) USE OF A NOVEL SUBCUTANEOUS NEEDLE-FREE TECHNIQUE TO DELIVER TESTOSTERONE IN HYPOGONADAL MEN

(71) Applicant: Bioject, Inc., Tigard, OR (US)

(72) Inventor: Mark A. LOGOMASINI, Lake Forest, CA (US)

(73) Assignee: Bioject, Inc., Tigard, OR (US)

(21) Appl. No.: 14/105,877

(22) Filed: Dec. 13, 2013

Related U.S. Application Data
(60) Provisional application No. 61/737,445, filed on Dec. 14, 2012.

(51) Int. Cl. A61M 5/30; A61K 31/568

(52) U.S. Cl. CPC A61M 5/30; A61K 31/568
USPC 604/522; 514/178

(57) ABSTRACT

Embodiments herein are directed to methods for increasing testosterone levels in a subject in need thereof, comprising administering to the subject a therapeutically effective amount of testosterone by needle-free injection to an injection site. Some embodiments are further directed to a method of increasing testosterone levels in a subject in need thereof, by administering to the subject a pharmaceutical composition comprising a therapeutically effective amount of testosterone and a pharmaceutically acceptable carrier by needle-free injection. Some embodiments are directed to a method of minimizing fluctuations in testosterone levels in a subject diagnosed with hypogonadism, comprising serially administering to the subject a therapeutically effective amount of testosterone by needle-free injection to an injection site.

### CROSS-REFERENCE TO RELATED APPLICATIONS

This application claims the benefit of U.S. Provisional Application No. 61/737,445 entitled “Use of a novel subcutaneous needle-free technique to deliver testosterone in hypogonadal men” filed Dec. 14, 2012, which is hereby incorporated by reference in its entirety.

### GOVERNMENT INTERESTS

Not Applicable

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

Not Applicable

### SUMMARY

Embodiments herein are directed to methods of increasing testosterone levels in a subject in need thereof, the method comprising administering to the subject a therapeutically effective amount of testosterone by needle-free injection to an injection site. In some embodiments, administering to the subject a therapeutically effective amount of testosterone by needle-free injection comprises administering the testosterone using a needle-free injection device. In some embodiments, the needle-free injection device comprises a ZetaJet delivery system. In some embodiments, the needle-free injection device is selected from a spring-powered injection device, a gas powered injection device and combinations thereof.

In some embodiments, the injection site is the abdomen, chest, back, buttocks, face, neck, upper arms, lower arms, upper legs, lower legs, hands, feet, groin, pubic area, or external sexual organs. In some embodiments, the injection site is not directly over a blood vessel.

In some embodiments, the subject is receiving testosterone through needle injection and wherein needle injection is substituted with needle-free injection.

In some embodiments, the subject is refractory to testosterone therapy

In some embodiments, administering to the subject a therapeutically effective amount of testosterone by needle-free injection is performed at least once every 168 hours. In some embodiments, administering to the subject a therapeutically effective amount of testosterone by needle-free injection is performed once every 24 hours. In some embodiments, administering to the subject a therapeutically effective amount of testosterone by needle-free injection is performed once every 48 hours. In some embodiments, administering to the subject a therapeutically effective amount of testosterone by needle-free injection is performed once every 48 hours with a 72 hour gap after three sequential administrations.

In some embodiments, administering to the subject a therapeutically effective amount of testosterone by needle-free injection delivers testosterone subcutaneously. In some embodiments, administering to the subject a therapeutically effective amount of testosterone by needle-free injection delivers testosterone intradermally. In some embodiments, administering to the subject a therapeutically effective amount of testosterone by needle-free injection delivers testosterone intramuscularly.

In some embodiments, the subject self-administers the testosterone by needle-free injection. In some embodiments, the testosterone by needle-free injection is administered by a medical professional.

In some embodiments, a therapeutically effective amount of testosterone is from about 1 mg to about 100 mg. In some embodiments, a therapeutically effective amount of testosterone is about 25 mg. In some embodiments, a therapeutically effective amount of testosterone is about 37.5 mg. In some embodiments, a therapeutically effective amount of testosterone is about 50 mg.

In some embodiments, the volume of testosterone administered by needle-free injection is from about 0.05 mL to about 1 mL. In some embodiments, the volume of testosterone administered by needle-free injection is from about 0.5 mL.

In some embodiments, the testosterone levels of the subject 6 hours after administering to the subject a therapeutically effective amount of testosterone by needle-free injection are from about 300 ng/dL to about 900 ng/dL.

In some embodiments, the subject is a human. In some embodiments, the subject is a human male. In some embodiments, the subject is a human male from about 40 to about 70 years of age. In some embodiments, the subject is clinically diagnosed with secondary hypogonadism. In some embodiments, the subject is a human male from about 40 to about 70 years of age clinically diagnosed with secondary hypogonadism. In some embodiments, the subjects serum total testosterone level is known. In some embodiments, the subject has a serum total testosterone level below about 300 ng/dL. In some embodiments, the subject has had a serum total testosterone level below about 300 ng/dL on at least two separate occasions prior to administration. In some embodiments, the subject is not taking a testosterone supplement, a testosterone pharmaceutical, a corticosteroid, a growth hormone supplement, dehydroepiandrosterone (DHEA), and luteinizing hormone-releasing hormone (LHRH) agonist or a combination thereof. In some embodiments, the subject is not pregnant. In some embodiments, the subject does not have a history of prostate cancer, a current diagnosis of prostate cancer or a combination thereof. In some embodiments, the subject has had a prior Testopel insertion and wherein at least one testosterone level in the L range and 5 months have passed since insertion.

In some embodiments, administering to the subject a therapeutically effective amount of testosterone by needle-free injection causes less pain to the subject than administration of testosterone by needle injection. In some embodiments, administering to the subject a therapeutically effective amount of testosterone by needle-free injection is more tolerable to the subject than administration of testosterone by needle injection. In some embodiments, administering to the subject a therapeutically effective amount of testosterone by needle-free injection results in less post injection wetness than administering testosterone by needle injection. In some embodiments, administering to the subject a therapeutically effective amount of testosterone by needle-free injection results in less redness, bruising, induration, or a combination thereof than administering testosterone by needle injection. In some embodiments, administering to the subject a therapeutically effective amount of testosterone by needle-free injection results in greater dispersion than administration with a needle injection. In some embodiments, administering to the subject a therapeutically effective amount of testosterone by needle-free injection does not cause damage to skin cells at the site of injection.

Some embodiments are directed to a method of increasing testosterone levels in a subject in need thereof, the method comprising administering to the subject a pharmaceutical composition comprising a therapeutically effective amount of testosterone and a pharmaceutically acceptable carrier by needle-free injection.

Some embodiments are directed to a method of delivering testosterone to a subject in need thereof, the method comprising administering to the subject a pharmaceutical composition comprising a therapeutically effective amount of testosterone and a pharmaceutically acceptable carrier by needle-free injection.

Some embodiments are directed to a method of treating hypogonadism or the symptoms thereof, the method comprising administering to a subject in need thereof a therapeutically effective amount of testosterone by a needle-free injection.

In some embodiments, the subject is refractory to testosterone therapy.

Some embodiments are directed to a method of treating secondary hypogonadism or the symptoms thereof in a subject in need thereof, the method comprising administering to the subject a therapeutically effective amount of testosterone by a needle-free injection.

In some embodiments, the subject is refractory to testosterone therapy.

Some embodiments are directed to a method of administering a hormone to a subject in need thereof, the method comprising administering the hormone via a needle-free injection to an injection site with a needle-free injection device.

In some embodiments, the needle-free injection device comprises a portable injector and a disposable syringe. In some embodiments, the disposable syringe is pre-loaded with a therapeutically effective amount of the hormone. In some embodiments, the hormone is testosterone. In some embodiments, the disposable syringe can be loaded with a variable amount of the hormone.

Some embodiments are directed to a method of minimizing fluctuations in testosterone levels in a subject diagnosed with hypogonadism, the method comprising: serially administering to the subject a therapeutically effective amount of testosterone by needle-free injection to an injection site; wherein the subjects' testosterone levels are maintained within a range from between about 300 ng/dL and about 900 ng/dL. In some embodiments, serially administering to the subject a therapeutically effective amount of testosterone comprises at least one injection every 168 hours. In further embodiments of the method, serially administering to the subject a therapeutically effective amount of testosterone comprises at least one injection every 24 hours. In some embodiments, serially administering to the subject a therapeutically effective amount of testosterone comprises at least one injection every 48 hours. In some embodiments, administering to the subject a therapeutically effective amount of testosterone by needle-free injection is performed once every 48 hours with a 72 hour gap after three sequential administrations.

### DESCRIPTION OF DRAWINGS

FIG. 1 depicts the average pain 5 minutes after injection in all patients studied.

FIG. 2 depicts the average pain 30 minutes after injection in all patients studied.

FIG. 3 depicts the average testosterone levels (ng/dl) in 14 men.

FIG. 4 depicts pre- and post-study AMS ratings by patient and as the average of all patients in the study.

FIG. 5 depicts post injection site reactions as a percentage of the total number of injections for 14 patients over a 30 day period.

FIG. 6 depicts post injection reaction occurrence for all patients over a thirty day period.

FIG. 7 depicts cumulative wetness results by category as a percentage of total injections.

FIG. 8 depicts the overall impression of needle-free injection.

FIG. 9 depicts patient's likelihood of using needle-free injection over needle and syringe.

### DETAILED DESCRIPTION

In the present disclosure, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figure, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

While various compositions, methods and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a”, “an”, or “the” limits any particular claim containing such introduced claim recitation to embodiments containing only one such recitation, even when the same claim includes the introductory phrases “one or more” or “at least one” and indefinite articles such as “a”, “an”, or “the” (e.g., “a” and/or “an” and/or “the” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges, which can be subsequently broken down into subranges as discussed above. Finally, as will be understood by one skilled in the art, a range includes each individual member. Thus, for example, a group having 1-3 substituents refers to groups having 1, 2, or 3 substituents. Similarly, a group having 1-5 substituents refers to groups having 1, 2, 3, 4, or 5 substituents, and so forth.

Age-related hormonal decline in males is gradual and less recognized than in females. Symptoms are often non-specific, causing difficulty for males to recognize, and oftentimes they are ignored. Hypogonadism symptoms in males include but are not limited to: fatigue, lack of concentration, mood swings, decreased sexual desire, erectile dysfunction, infertility, hair loss, reduced muscle and bone mass, weight gain or a combination thereof. Male hypogonadism has been linked to reduction in quality of life, and poorer health outcomes as it may increase the risk for cardiovascular disease, diabetes mellitus, metabolic syndrome, Alzheimer's disease, and premature death.

In addition to oral and injectable testosterone, existing formulations consist of topical or transdermal testosterone replacement therapies, including patches and gels. Although these approaches yield near-physiologic concentrations of testosterone, they are expensive and are hindered by variable absorption across the skin and possible transference to family members. Restoration of testosterone levels to the eugonadal range reverses the signs and symptoms of hypogonadism, except for infertility, and may alleviate co-morbidities associated with hypogonadism. Patient compliance and understanding of the treatment along with monitoring are of utmost importance to achieve clinical success with maximum benefit and minimum risk.

There are practical and clinical drawbacks to each of the different ways to administer testosterone: intramuscular can generate too much fluctuation in testosterone levels and often requires bi-weekly visits to the physician clinic. Gels and creams warn against touching other family members to prevent unwanted transference, and also require time to allow for absorption and pharmacokinetics can vary by climate and varying body habitus.

Subcutaneous daily administration may allow home use for males to quickly administer and allow for consistent daily levels. Subcutaneous administration may be a safe and feasible alternative to administering testosterone when compared to conventional IM injection or dermal absorption. In some embodiments, subcutaneous needle-free injection represents an alternative form of testosterone delivery that provides the benefits of subcutaneous administration, but with decreased pain and side effects along with an increased ease of use than current treatment modalities would be highly beneficial for hypogonadal subjects.

There is a need for an alternative form of testosterone delivery that provides reduced side effects and decreased pain along with an increased ease of use compared with current treatment modalities. In some embodiments, a needle-free device for testosterone delivery may result in decreased pain and increased ease of use compared with current treatment modalities for conditions associated with abnormal hormone levels. In some embodiments, a needle-free device for testosterone delivery may result in decreased pain and increased ease of use compared with current treatment modalities for conditions associated with abnormal testosterone levels. In some embodiments, a needle-free device for testosterone delivery may result in decreased pain and increased ease of use compared with current treatment modalities and be highly beneficial for hypogonadal males. In some embodiments, a needle-free device for testosterone delivery will also be suitable for subjects such as, but not limited to transsexual adolescents who have delayed puberty and fulfill the eligibility to begin male puberty, and female-to-male transsexual persons who undergo hormone replacement.

Some embodiments are directed to a method of increasing testosterone levels in a subject in need thereof, the method comprising administering to the subject a therapeutically effective amount of testosterone by needle-free injection.

In some embodiments, needle-free injection can deliver liquids across the skin by the use of jet injection, forcing the liquid at high speed through a tiny orifice held against the skin. In some embodiments, needle-free injection causes a fine stream of high-pressure liquid to penetrate the skin and deposit the liquid in the tissue beneath. In some embodiments, the orifice and the pressure are adjustable, allowing the device to deliver various precise doses of medication to specific depths accurately and consistently. In some embodiments, the liquid may contain a medication, in some embodiments, the medication is a hormone. In some embodiments, the medication is testosterone.

In some embodiments, needle-free injection is a safe and effective alternative administration of many different medications for a variety of applications. In some embodiments, needle-free injection can be used for immunization and mass inoculations. In some embodiments, vaccination of large populations with needle-free injections can be carried out safely and effectively.

In some embodiments, administering to the subject a therapeutically effective amount of testosterone by needle-free injection comprises administering the testosterone using a needle-free injection device. In some embodiments, the needle-free injection device is selected from a spring-powered injection device, a gas powered injection device and combinations thereof. In some embodiments, the needle-free injection device comprises the Biojector 2000 or the Zetajet. In some embodiments, needle-free injection is achieved using a spring-powered jet injector. In some embodiments, examples of spring powered jet injectors include, but are not limited to the Biojector B2000 and the Zetajet. In some embodiments, needle-free injection is achieved using a gas powered jet injector. In some embodiments, examples of gas powered jet injectors include but are not limited to the Biojector B2000. In some embodiments, the pressure profile of the Zetajet is virtually the same as that of the B2000. In some embodiments, SQ, intradermal, and IM injections with the B2000 and the Zetajet are equivalent between the two devices. In some embodiments, the Zetajet has equivalent performance characteristics as the B2000.

In some embodiments, the Zetajet uses sterile, single use syringes for individual injections to prevent cross-contamination shown with fixed-nozzle jet injection systems. Clinical studies on jet injection in combination with magnetic resonance imaging studies on drug dispersion patterns suggest that the Zetajet is a valid replacement of the needle and syringe for many subcutaneous or IM injections. Both the Biojector and Zetajet have been cleared by the FDA for subcutaneous and intramuscular injections of vaccines and other injectable drugs.

In some embodiments, the needle-free injection device comprises a portable injector and a disposable syringe. In other embodiments of the method, the needle-free injection device comprises a portable injector and a non-disposable syringe. In some embodiments, the disposable syringe is pre-loaded with a therapeutically effective amount of the hormone. In some embodiments, the disposable syringe is not pre-loaded with a therapeutically effective amount of the hormone. In some embodiments, the disposable syringe can be loaded with a variable amount of the hormone. In some embodiments, the disposable syringe can also not be pre-loaded with a therapeutically effective amount of the hormone. In some embodiments, the hormone is testosterone.

In some embodiments, testosterone can be delivered to various areas of the body, including but not limited to the abdomen, chest, back, buttocks, face, neck, upper arms, lower arms, upper legs, lower legs, hands, feet, groin, pubic area, or external sexual organs. In some embodiments, the injection site is any part of the body suitable for injection. In some embodiments, the injection site is any site on the body that would be suitable for a traditional needle injection. In some embodiments, the injection site is the abdomen, chest, back, buttocks, face, neck, upper arms, lower arms, upper legs, lower legs, hands, feet, groin, pubic area, or external sexual organs. In some embodiments, the injection site is not directly over a blood vessel.

In some embodiments, the subject is receiving testosterone through needle injection and needle injection is substituted with needle-free injection. In some embodiments, the subject is already receiving testosterone via a conventional method such as but not limited to needle injection, a gel, a patch or a combination thereof. In some embodiments, the subject is refractory to testosterone treatments. As used herein, the term “refractory” is intended to mean that a subject, when given a testosterone treatment to treat hypogonadism, does not exhibit a response characterized by an increase in blood testosterone levels above about 300 ng/dl. In some embodiments, the increase in blood testosterone levels will be determined based on the age of the subject, or will be determined by a clinician. In some embodiments, the subject is refractive to a topical testosterone therapy used to treat hypogonadism such as, but not limited to testosterone creams and gels. In some embodiments, the subject is refractory to an injectable testosterone therapy used to treat hypogonadism such as, but not limited to injectable pellets. In yet other embodiments the subject is refractory to all testosterone treatments used to treat hypogonadism.

In some embodiments, the subject is administered a therapeutically effective amount of testosterone by needle-free injection. In some embodiments, administering to the subject a therapeutically effective amount of testosterone by needle-free injection once every 12 hours. In some embodiments, administering to the subject a therapeutically effective amount of testosterone by needle-free injection once every 24 hours. In some embodiments, administering to the subject a therapeutically effective amount of testosterone by needle-free injection once every 36 hours. In some embodiments, administering to the subject a therapeutically effective amount of testosterone by needle-free injection once every 48 hours. In some embodiments, the administering to the subject a therapeutically effective amount of testosterone by needle-free injection once every 60 hours. In some embodiments, administering to the subject a therapeutically effective amount of testosterone by needle-free injection once every 72 hours. In some embodiments, administering to the subject a therapeutically effective amount of testosterone by needle-free injection once every 168 hours. In some embodiments, administering to the subject a therapeutically effective amount of testosterone by needle-free injection once every 180 hours. In some embodiments, administering to the subject a therapeutically effective amount of testosterone by needle-free injection at any time between any of these values. In some embodiments, administering to the subject a therapeutically effective amount of testosterone by needle-free injection is performed on a weekly administration cycle wherein administering to the subject a therapeutically effective amount of testosterone by needle-free injection is performed three times a week on day 1 (0 hours), day 3 (48 hours), and day 5 (96 hours). In some embodiments, administering to the subject a therapeutically effective amount of testosterone by needle-free injection is performed on a weekly administration cycle wherein administration is performed at 0 hours, 48 hours and 96 hours after which no administration is given for 72 hours after which the cycle re-starts. For example, administering to the subject a therapeutically effective amount of testosterone by needle-free injection may be performed on a Monday, Wednesday, and Friday after the administration cycle restarts on the following Monday.

In some embodiments, administering to the subject a therapeutically effective amount of testosterone by needle-free injection at least once every 12 hours. In some embodiments, administering to the subject a therapeutically effective amount of testosterone by needle-free injection at least once every 24 hours. In some embodiments, administering to the subject a therapeutically effective amount of testosterone by needle-free injection at least once every 36 hours. In some embodiments, administering to the subject a therapeutically effective amount of testosterone by needle-free injection at least once every 48 hours. In some embodiments, the administering to the subject a therapeutically effective amount of testosterone by needle-free injection at least once every 60 hours. In some embodiments, administering to the subject a therapeutically effective amount of testosterone by needle-free injection at least once every 72 hours. In some embodiments, administering to the subject a therapeutically effective amount of testosterone by needle-free injection at least once every 168 hours. In some embodiments, administering to the subject a therapeutically effective amount of testosterone by needle-free injection at least once every 180 hours. In some embodiments, administering to the subject a therapeutically effective amount of testosterone by needle-free injection at any time between any of these values.

In some embodiments, administering to the subject a therapeutically effective amount of testosterone by needle-free injection delivers testosterone subcutaneously. In some embodiments, testosterone administration can be delivered subcutaneously to various areas of the body, including but not limited to the abdomen, chest, back, buttocks, face, neck, upper arms, lower arms, upper legs, lower legs, hands, feet, groin, pubic area, or external sexual organs. In some embodiments, administering to the subject a therapeutically effective amount of testosterone by needle-free injection delivers testosterone intradermally. In some embodiments, testosterone administration can be delivered intradermally to various areas of the body, including but not limited to the abdomen, chest, back, buttocks, face, upper arms, or upper legs. In some embodiments, administering to the subject a therapeutically effective amount of testosterone by needle-free injection delivers testosterone intramuscularly. In some embodiments, testosterone administration can be delivered intramuscularly to various areas of the body, including but not limited to the abdomen, chest, back, buttocks, face, neck, upper arms, lower arms, upper legs, lower legs, hands, feet, groin, pubic area, or external sexual organs. In some embodiments, the subject can be administered a therapeutically effective amount of testosterone by needle-free injection which is delivered by any other means of penetration of the skin or combinations thereof. In some embodiments, testosterone administration can be delivered to various areas of the body, including but not limited to the abdomen, chest, back, buttocks, face, neck, upper arms, lower arms, upper legs, lower legs, hands, feet, groin, pubic area, or external sexual organs.

In some embodiments, the subject self-administers the testosterone by needle-free injection. In some embodiments, the subject self-administers the testosterone by needle-free injection after training by a medical professional. In some embodiments, the subject will forego the necessity of clinical visits for each injection, offering reduced cost and time expenditures. In some embodiments, self-administration also provides the ability for the subject to administer the injection at any location. In some embodiments, the testosterone by needle-free injection is administered by a medical professional.

In some embodiments, a therapeutically effective amount of testosterone is about 1 mg to about 100 mg. In some embodiments, a therapeutically effective amount of testosterone is about 25 mg. In some embodiments, a therapeutically effective amount of testosterone is about 37.5 mg. In some embodiments, a therapeutically effective amount of testosterone is about 50 mg. In some embodiments, a therapeutically effective amount of testosterone is about 0.1 mg to about 150 mg, and a value between any of those values. In some embodiments, a therapeutically effective amount may be assessed in any manner known in the art, including but not limited to determining a subject as no longer hypogonadal. In some embodiments, a therapeutically effective amount can assessed by the amelioration of at least one symptom of hypogonadism. In some embodiments, symptoms of hypogonadism include, but are not limited to, fatigue, lack of concentration, mood swings, decreased sexual desire, erectile dysfunction, infertility, hair loss, reduced muscle and bone mass, weight gain or a combination thereof.

In some embodiments, one of ordinary skill in the art will understand and appreciate the dosages and timing of said dosages to be administered to a patient in need thereof. The doses and duration of treatment may vary, and may be based on assessment by one of ordinary skill in the art based on monitoring and measuring the symptoms of hypogonadism, testosterone levels or a combination thereof. This assessment may be made based on outward physical signs of hypogonadism, such as but not limited to fatigue, lack of concentration, mood swings, decreased sexual desire, erectile dysfunction, infertility, hair loss, reduced muscle and bone mass, weight gain or a combination thereof. The doses may also depend on the condition or disease being treated, the degree of the condition or disease being treated and further on the age and weight of the patient.

Specific modes of administration will depend on the indication. The selection of the specific route of administration and the dose regimen may be adjusted or titrated by the clinician according to methods known to the clinician in order to obtain the optimal clinical response. The amount of compound to be administered may be that amount which is therapeutically effective. The dosage to be administered may depend on the characteristics of the subject being treated, e.g., the particular animal or human subject treated, age, weight, health, types of concurrent treatment, if any, and frequency of treatments, and can be easily determined by one of skill in the art (e.g., by the clinician).

In some embodiments, the volume of testosterone administered by needle-free injection is from about 0.05 mL to about 1 mL, and any value in between these two values. In some embodiments, the volume of testosterone administered by needle-free injection is about 0.5 mL.

In some embodiments, the administration of a therapeutically effective amount of testosterone to a subject results in an increase in testosterone levels in the subject. In some embodiments, the testosterone levels of the subject 6 hours after administering to the subject a therapeutically effective amount of testosterone by needle-free injection are from about 300 ng/dL to about 900 ng/dL, from about 400 ng/dL to about 900 ng/dL, from about 500 ng/dL to about 900 ng/dL, from about 600 ng/dL to about 900 ng/dL, from about 700 ng/dL to about 900 ng/dL, from about 800 ng/dL to about 900 ng/dL, or a range between any two of these values.

In some embodiments, the subject is a human. In some embodiments, the subject is a human male. In embodiments, the subject is a human female. In some embodiments, the subject is not pregnant. In some embodiments, the subject is a human male is from about 40 to about 70 years of age. In some embodiments, the subject is clinically diagnosed with hypogonadism. In some embodiments, the subject is clinically diagnosed with secondary hypogonadism. In some embodiments, the subject is a human male from about 40 to about 70 years of age clinically diagnosed with secondary hypogonadism. In some embodiments, the subject is refractory to testosterone treatments. In some embodiments, the increase in blood testosterone levels will be determined based on the age of the subject, or will be determined by a physician. In some embodiments, the subject is refractive to a topical testosterone therapy used to treat hypogonadism such as, but not limited to testosterone creams and gels. In some embodiments, the subject is refractory to an injectable testosterone therapy used to treat hypogonadism such as, but not limited to injectable pellets. In yet other embodiments the subject is refractory to all testosterone treatments used to treat hypogonadism. In some embodiments, the subject is a hypogonadal transsexual adolescent. In some embodiments, the subject is a hypogonadal transsexual adolescent receiving testosterone for induction of male puberty. In some embodiments, the subject is a transsexual adult. In some embodiments, the subject is a transsexual adult receiving hormone replacement treatment using the same principals for the treatment of hypogonadal subjects.

In some embodiments, while administering testosterone can be for humans, those of ordinary skill in the art recognize that veterinary uses may also apply.

In some embodiments, the subjects serum total testosterone level is known. In some embodiments, the subject has a serum total testosterone level below about 300 ng/dL. In some embodiments, the subject has had a serum total testosterone level below about 300 ng/dL on at least two separate occasions prior to administration. In some embodiments, the subject has had a serum total testosterone level below about 400 ng/dL, below about 500 ng/dL, below a detectable amount or a combination thereof.

In some embodiments, the subject is not taking a testosterone supplement, a testosterone pharmaceutical, a corticosteroid, a growth hormone supplement, DHEA, LHRH agonist or a combination thereof. In some embodiments, the subject does not have a history of prostate cancer, a current diagnosis of prostate cancer or a combination thereof. In some embodiments, the subject has had a prior Testopel insertion and wherein at least one testosterone level in the L range and 5 months have passed since insertion.

In some embodiments, administering to a subject a therapeutically effective amount of testosterone by needle-free injection causes less pain to the subject than administration of testosterone by needle-injection. In some embodiments, in order to assess pain level during treatment, any index known in the art may be used, including but not limited to a visual analog pain scale from 0-10, a verbal pain scale from 0-4 or a combination thereof.

In some embodiments, administering to the subject a therapeutically effective amount of testosterone by needle-free injection is more tolerable to the subject than administration of testosterone by needle injection.

In some embodiments, administering to the subject a therapeutically effective amount of testosterone by needle-free injection results in less post injection wetness than administering testosterone by needle injection.

In some embodiments, administering to the subject a therapeutically effective amount of testosterone by needle-free injection results in less redness, bruising, induration, or a combination thereof than administering testosterone by needle injection.

In some embodiments, administering to the subject a therapeutically effective amount of testosterone by needle-free injection results in greater dispersion than administration with a needle injection.

In some embodiments, administering to the subject a therapeutically effective amount of testosterone by needle-free injection does not cause damage to skin cells at the site of injection.

Some embodiments are directed to a method of delivering testosterone to a subject in need thereof, the method comprising administering to the subject a pharmaceutical composition comprising a therapeutically effective amount of testosterone and a pharmaceutically acceptable carrier by needle-free injection.

In some embodiments, the subject is administered a pharmaceutical composition comprising a therapeutically effective amount of testosterone and a pharmaceutically acceptable carrier by needle-free injection.

In some embodiments, the subject is administered a pharmaceutical composition by needle-free injection once every 12 hours. In some embodiments, the subject is administered a pharmaceutical composition by needle-free injection once every 24 hours. In some embodiments, the subject is administered a pharmaceutical composition by needle-free injection once every 36 hours. In some embodiments, the subject is administered a pharmaceutical composition by needle-free injection once every 48 hours. In some embodiments, the subject is administered a pharmaceutical composition by needle-free injection once every 60 hours. In some embodiments, the subject is administered a pharmaceutical composition by needle-free injection once every 72 hours. In some embodiments, the subject is administered a pharmaceutical composition by needle-free injection once every 168 hours. In some embodiments, the subject is administered a pharmaceutical composition by needle-free injection once every 180 hours. In some embodiments, the subject is administered a pharmaceutical composition by needle-free injection at any time between any of these values. In some embodiments, administering to the subject a therapeutically effective amount of testosterone by needle-free injection is performed on a weekly administration cycle wherein administering to the subject a therapeutically effective amount of testosterone by needle-free injection is performed three times a week on day 1 (0 hours), day 3 (48 hours), and day 5 (96 hours). In some embodiments, administering to the subject a therapeutically effective amount of testosterone by needle-free injection is performed on a weekly administration cycle wherein administration is performed at 0 hours, 48 hours and 96 hours after which no administration is given for 72 hours after which the cycle re-starts. For example, administering to the subject a therapeutically effective amount of testosterone by needle-free injection may be performed on a Monday, Wednesday, and Friday after the administration cycle restarts on the following Monday.

In some embodiments, administering to the subject a pharmaceutical composition by needle-free injection at least once every 12 hours. In some embodiments, administering to the subject a pharmaceutical composition by needle-free injection at least once every 24 hours. In some embodiments, administering to the subject a pharmaceutical composition by needle-free injection at least once every 36 hours. In some embodiments, administering to the subject a pharmaceutical composition by needle-free injection at least once every 48 hours. In some embodiments, the administering to the subject a pharmaceutical composition by needle-free injection at least once every 60 hours. In some embodiments, administering to the subject a pharmaceutical composition by needle-free injection at least once every 72 hours. In some embodiments, administering to the subject a pharmaceutical composition by needle-free injection at least once every 168 hours. In some embodiments, administering to the subject a pharmaceutical composition by needle-free injection at least once every 180 hours. In some embodiments, administering to the subject a pharmaceutical composition by needle-free injection at any time between any of these values.

In some embodiments, administering to the subject a pharmaceutical composition by needle-free injection delivers testosterone subcutaneously. In some embodiments, a pharmaceutical composition is delivered subcutaneously by needle-free injection to various areas of the body, including but not limited to the abdomen, chest, back, buttocks, face, neck, upper arms, lower arms, upper legs, lower legs, hands, feet, groin, pubic area, or external sexual organs. In some embodiments, a pharmaceutical composition is delivered intradermally by needle-free injection. In some embodiments, a pharmaceutical composition is delivered intradermally by needle-free injection to various areas of the body, including but not limited to the abdomen, chest, back, buttocks, face, neck, upper arms, lower arms, upper legs, lower legs, hands, feet, groin, pubic area, or external sexual organs. In some embodiments, a pharmaceutical composition is delivered intramuscularly by needle-free injection. In some embodiments, a pharmaceutical composition is delivered intramuscularly by needle-free injection to various areas of the body, including but not limited to the abdomen, chest, back, buttocks, face, neck, upper arms, lower arms, upper legs, lower legs, hands, feet, groin, pubic area, or external sexual organs. In some embodiments, the subject can be administered a pharmaceutical composition by needle-free injection which is delivered by any other means of penetration of the skin. In some embodiments, a pharmaceutical composition delivered by needle-free injection can be delivered to various areas of the body, including but not limited to the abdomen, chest, back, buttocks, face, neck, upper arms, lower arms, upper legs, lower legs, hands, feet, groin, pubic area, or external sexual organs.

In some embodiments, the subject self-administers the pharmaceutical composition by needle-free injection. In some embodiments, the subject self-administers the pharmaceutical composition by needle-free injection after training by a medical professional. In some embodiments, the subject will forego the necessity of clinical visits for each injection, offering reduced cost and time expenditures. In some embodiments, self-administration also provides the ability for the subject to administer the injection at any location. In some embodiments, the testosterone by needle-free injection is administered by a medical professional.

In some embodiments, a therapeutically effective amount of testosterone in a pharmaceutical composition is about 1 mg to about 100 mg. In some embodiments, a therapeutically effective amount of testosterone in a pharmaceutical composition is about 25 mg. In some embodiments, a therapeutically effective amount of testosterone in a pharmaceutical composition is about 37.5 mg. In some embodiments, a therapeutically effective amount of testosterone is about 50 mg. In some embodiments, a therapeutically effective amount of testosterone in a pharmaceutical composition is about 0.1 mg to about 150 mg, and a value between any of those values. In some embodiments, a therapeutically effective amount of testosterone in a pharmaceutical composition may be assessed in any manner known in the art, including but not limited to determining a subject as no longer hypogonadal. In some embodiments, a therapeutically effective in a pharmaceutical composition amount can assessed by the amelioration of at least one symptom of hypogonadism. In some embodiments, symptoms of hypogonadism include, but are not limited to, fatigue, lack of concentration, mood swings, decreased sexual desire, erectile dysfunction, infertility, hair loss, reduced muscle and bone mass, weight gain or a combination thereof.

In some embodiments, the pharmaceutical composition comprises testosterone cypionate. In some embodiments, the pharmaceutical composition comprises a therapeutically effective amount of testosterone cypionate. In some embodiments, the pharmaceutical compositions further comprise benzyl benzoate, cottonseed oil, benzyl alcohol and any combination thereof.

In some embodiments, the pharmaceutical compositions can be formulated with a suitable carrier for needle-free injection in a variety of dosage forms including, but not limited to, solutions, powders, emulsions, suspensions, semi-solids, ointments, pastes, creams, gels and jellies, foams, and dry powders. It is also known in the art that the active ingredients can be contained in such formulations with pharmaceutically acceptable diluents, fillers, disintegrants, binders, lubricants, surfactants, hydrophobic vehicles, water-soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

In some embodiments, pharmaceutical compositions can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, pharmaceutical compositions can be formulated readily by combining these compounds with pharmaceutically acceptable carriers well known in the art. As used herein, the term “pharmaceutically acceptable carrier” means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers include but are not limited to sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Such carriers enable the compounds of the invention to be formulated as liquids, gels, syrups, slurries, suspensions and the like, for needle-free injection. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

In some embodiments, pharmaceutical compositions comprise aqueous suspensions. Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, and one or more coloring agents.

Pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

Pharmaceutical compositions can also be administered in combination with other active ingredients, such as, for example, adjuvants, protease inhibitors, or other compatible drugs or compounds where such combination is seen to be desirable or advantageous in achieving the desired effects of the methods described herein.

Some embodiments are directed to a method of treating hypogonadism or the symptoms thereof, the method comprising administering to the subject in need thereof a therapeutically effective amount of testosterone by a needle-free injection.

In some embodiments, treating hypogonadism comprises administering to the subject a pharmaceutical composition comprising a therapeutically effective amount of testosterone and a pharmaceutically acceptable carrier by needle-free injection.

In some embodiments, treating hypogonadism comprises increasing testosterone levels in a subject. In some embodiments, treating testosterone comprises increasing testosterone levels in a subject to a range of about 300 ng/dL to about 900 ng/dL.

In some embodiments, treating hypogonadism comprises ameliorating one or more of the symptoms of hypogonadism. In some embodiments, the symptoms of hypogonadism include, but are not limited to, fatigue, lack of concentration, mood swings, decreased sexual desire, erectile dysfunction, infertility, hair loss, reduced muscle and bone mass, weight gain or a combination thereof.

In some embodiments, the subject is administered a therapeutically effective amount of testosterone by needle-free injection. In some embodiments, the subject is administered a therapeutically effective amount of testosterone by needle-free injection once every 12 hours. In some embodiments, the subject is administered a therapeutically effective amount of testosterone by needle-free injection once every 24 hours. In some embodiments, the subject is administered a therapeutically effective amount of testosterone by needle-free injection once every 36 hours. In some embodiments, the subject is administered a therapeutically effective amount of testosterone by needle-free injection once every 48 hours. In some embodiments, the subject is administered a therapeutically effective amount of testosterone by needle-free injection once every 60 hours. In some embodiments, the subject is administered a therapeutically effective amount of testosterone by needle-free injection once every 72 hours. In some embodiments, the subject is administered a therapeutically effective amount of testosterone by needle-free injection once every 168 hours. In some embodiments, the subject is administered a therapeutically effective amount of testosterone by needle-free injection once every 180 hours. In some embodiments, the subject is administered a therapeutically effective amount of testosterone by needle-free injection at any time between any of these values. In some embodiments, administering to the subject a therapeutically effective amount of testosterone by needle-free injection is performed on a weekly administration cycle wherein administering to the subject a therapeutically effective amount of testosterone by needle-free injection is performed three times a week on day 1 (0 hours), day 3 (48 hours), and day 5 (96 hours). In some embodiments, administering to the subject a therapeutically effective amount of testosterone by needle-free injection is performed on a weekly administration cycle wherein administration is performed at 0 hours, 48 hours and 96 hours after which no administration is given for 72 hours after which the cycle re-starts. For example, administering to the subject a therapeutically effective amount of testosterone by needle-free injection may be performed on a Monday, Wednesday, and Friday after the administration cycle restarts on the following Monday.

In some embodiments, administering to the subject a therapeutically effective amount of testosterone by needle-free injection at least once every 12 hours. In some embodiments, administering to the subject a therapeutically effective amount of testosterone by needle-free injection at least once every 24 hours. In some embodiments, administering to the subject a therapeutically effective amount of testosterone by needle-free injection at least once every 36 hours. In some embodiments, administering to the subject a therapeutically effective amount of testosterone by needle-free injection at least once every 48 hours. In some embodiments, the administering to the subject a therapeutically effective amount of testosterone by needle-free injection at least once every 60 hours. In some embodiments, administering to the subject a therapeutically effective amount of testosterone by needle-free injection at least once every 72 hours. In some embodiments, administering to the subject a therapeutically effective amount of testosterone by needle-free injection at least once every 168 hours. In some embodiments, administering to the subject a therapeutically effective amount of testosterone by needle-free injection at least once every 180 hours. In some embodiments, administering to the subject a therapeutically effective amount of testosterone by needle-free injection at any time between any of these values.

In some embodiments, administering to the subject a therapeutically effective amount of testosterone by needle-free injection delivers testosterone subcutaneously. In some embodiments, testosterone administration can be delivered subcutaneously to various areas of the body, including but not limited to the abdomen, chest, back, buttocks, face, neck, upper arms, lower arms, upper legs, lower legs, hands, feet, groin, pubic area, or external sexual organs. In other embodiments of the method, administering to the subject a therapeutically effective amount of testosterone by needle-free injection delivers testosterone intradermally. In some embodiments, testosterone administration can be delivered intradermally to various areas of the body, including but not limited to the abdomen, chest, back, buttocks, face, neck, upper arms, lower arms, upper legs, lower legs, hands, feet, groin, pubic area, or external sexual organs. In further embodiments of the method, administering to the subject a therapeutically effective amount of testosterone by needle-free injection delivers testosterone intramuscularly. In some embodiments, testosterone administration can be delivered intramuscularly to various areas of the body, including but not limited to the abdomen, chest, back, buttocks, face, neck, upper arms, lower arms, upper legs, lower legs, hands, feet, groin, pubic area, or external sexual organs. In certain embodiments of the method, the subject can be administered a therapeutically effective amount of testosterone by needle-free injection which is delivered by any other means of penetration of the skin or combinations thereof. In some embodiments, testosterone administration can be delivered to various areas of the body, including but not limited to the abdomen, chest, back, buttocks, face, neck, upper arms, lower arms, upper legs, lower legs, hands, feet, groin, pubic area, or external sexual organs.

In some embodiments, the subject self-administers the testosterone by needle-free injection. In some embodiments, the subject self-administers the testosterone by needle-free injection after training by a medical professional. In some embodiments, the subject will forego the necessity of clinical visits for each injection, offering reduced cost and time expenditures. In some embodiments, Self-administration also provides the ability for the subject to administer the injection at any location. In some embodiments, the testosterone by needle-free injection is administered by a medical professional.

In some embodiments, a therapeutically effective amount of testosterone is about 1 mg to about 100 mg. In some embodiments, a therapeutically effective amount of testosterone is about 25 mg. In some embodiments, a therapeutically effective amount of testosterone is about 37.5 mg. In some embodiments, a therapeutically effective amount of testosterone is about 50 mg. In some embodiments, a therapeutically effective amount of testosterone is about 0.1 mg to about 150 mg, and a value between any of those values. In some embodiments, a therapeutically effective amount may be assessed in any manner known in the art, including but not limited to determining a subject as no longer hypogonadal. In some embodiments, a therapeutically effective amount can assessed by the amelioration of at least one symptom of hypogonadism. In some embodiments, symptoms of hypogonadism include, but are not limited to, fatigue, lack of concentration, mood swings, decreased sexual desire, erectile dysfunction, infertility, hair loss, reduced muscle and bone mass, weight gain or a combination thereof.

Some embodiments are directed to a method of treating secondary hypogonadism or the symptoms thereof where the method comprising administering to the subject a therapeutically effective amount of testosterone by a needle-free injection.

In some embodiments, treating secondary hypogonadism comprises administering to the subject a pharmaceutical composition comprising a therapeutically effective amount of testosterone and a pharmaceutically acceptable carrier by needle-free injection. In some embodiments, the subject is refractory to testosterone therapy. In some embodiments, the increase in blood testosterone levels will be determined based on the age of the subject, or will be determined by a physician. In some embodiments, the subject is refractive to a topical testosterone therapy used to treat hypogonadism such as, but not limited to testosterone creams and gels. In some embodiments, the subject is refractory to an injectable testosterone therapy used to treat hypogonadism such as, but not limited to injectable pellets. In yet other embodiments the subject is refractory to all testosterone treatments used to treat hypogonadism.

In some embodiments, the subject is administered a therapeutically effective amount of testosterone by needle-free injection. In some embodiments, the subject is administered a therapeutically effective amount of testosterone by needle-free injection once every 12 hours. In some embodiments, the subject is administered a therapeutically effective amount of testosterone by needle-free injection once every 24 hours. In some embodiments, the subject is administered a therapeutically effective amount of testosterone by needle-free injection once every 36 hours. In some embodiments, the subject is administered a therapeutically effective amount of testosterone by needle-free injection once every 48 hours. In some embodiments, the subject is administered a therapeutically effective amount of testosterone by needle-free injection once every 60 hours. In some embodiments, the subject is administered a therapeutically effective amount of testosterone by needle-free injection once every 72 hours. In some embodiments, the subject is administered a therapeutically effective amount of testosterone by needle-free injection once every 168 hours. In some embodiments, the subject is administered a therapeutically effective amount of testosterone by needle-free injection once every 180 hours. In some embodiments, the subject is administered a therapeutically effective amount of testosterone by needle-free injection at any time between any of these values. In some embodiments, administering to the subject a therapeutically effective amount of testosterone by needle-free injection is performed on a weekly administration cycle wherein administering to the subject a therapeutically effective amount of testosterone by needle-free injection is performed three times a week on day 1 (0 hours), day 3 (48 hours), and day 5 (96 hours). In some embodiments, administering to the subject a therapeutically effective amount of testosterone by needle-free injection is performed on a weekly administration cycle wherein administration is performed at 0 hours, 48 hours and 96 hours after which no administration is given for 72 hours after which the cycle re-starts. For example, administering to the subject a therapeutically effective amount of testosterone by needle-free injection may be performed on a Monday, Wednesday, and Friday after the administration cycle restarts on the following Monday.

In some embodiments, administering to the subject a therapeutically effective amount of testosterone by needle-free injection at least once every 12 hours. In some embodiments, administering to the subject a therapeutically effective amount of testosterone by needle-free injection at least once every 24 hours. In some embodiments, administering to the subject a therapeutically effective amount of testosterone by needle-free injection at least once every 36 hours. In some embodiments, administering to the subject a therapeutically effective amount of testosterone by needle-free injection at least once every 48 hours. In some embodiments, the administering to the subject a therapeutically effective amount of testosterone by needle-free injection at least once every 60 hours. In some embodiments, administering to the subject a therapeutically effective amount of testosterone by needle-free injection at least once every 72 hours. In some embodiments, administering to the subject a therapeutically effective amount of testosterone by needle-free injection at least once every 168 hours. In some embodiments, administering to the subject a therapeutically effective amount of testosterone by needle-free injection at least once every 180 hours. In some embodiments, administering to the subject a therapeutically effective amount of testosterone by needle-free injection at any time between any of these values.

In some embodiments, administering to the subject a therapeutically effective amount of testosterone by needle-free injection delivers testosterone subcutaneously. In some embodiments, testosterone administration can be delivered subcutaneously to various areas of the body, including but not limited to the abdomen, chest, back, buttocks, face, neck, upper arms, lower arms, upper legs, lower legs, hands, feet, groin, pubic area, or external sexual organs. In other embodiments of the method, administering to the subject a therapeutically effective amount of testosterone by needle-free injection delivers testosterone intradermally. In some embodiments, testosterone administration can be delivered intradermally to various areas of the body, including but not limited to the abdomen, chest, back, buttocks, face, neck, upper arms, lower arms, upper legs, lower legs, hands, feet, groin, pubic area, or external sexual organs. In further embodiments of the method, administering to the subject a therapeutically effective amount of testosterone by needle-free injection delivers testosterone intramuscularly. In some embodiments, testosterone administration can be delivered intramuscularly to various areas of the body, including but not limited to the abdomen, chest, back, buttocks, face, neck, upper arms, lower arms, upper legs, lower legs, hands, feet, groin, pubic area, or external sexual organs. In certain embodiments of the method, the subject can be administered a therapeutically effective amount of testosterone by needle-free injection which is delivered by any other means of penetration of the skin or combinations thereof. In some embodiments, testosterone administration can be delivered to various areas of the body, including but not limited to the abdomen, chest, back, buttocks, face, neck, upper arms, lower arms, upper legs, lower legs, hands, feet, groin, pubic area, or external sexual organs.

In some embodiments, the subject self-administers the testosterone by needle-free injection. In some embodiments, the subject self-administers the testosterone by needle-free injection after training by a medical professional. In some embodiments, the subject will forego the necessity of clinical visits for each injection, offering reduced cost and time expenditures. In some embodiments, self-administration also provides the ability for the subject to administer the injection at any location. In some embodiments, the testosterone by needle-free injection is administered by a medical professional.

In some embodiments, a therapeutically effective amount of testosterone is about 1 mg to about 100 mg. In some embodiments, a therapeutically effective amount of testosterone is about 25 mg. In some embodiments, a therapeutically effective amount of testosterone is about 37.5 mg. In some embodiments, a therapeutically effective amount of testosterone is about 0.1 mg to about 150 mg, and a value between any of those values. In some embodiments, a therapeutically effective amount may be assessed in any manner known in the art, including but not limited to determining a subject as no longer hypogonadal. In some embodiments, a therapeutically effective amount can assessed by the amelioration of at least one symptom of hypogonadism. In some embodiments, symptoms of hypogonadism include, but are not limited to, fatigue, lack of concentration, mood swings, decreased sexual desire, erectile dysfunction, infertility, hair loss, reduced muscle and bone mass, weight gain or a combination thereof.

In some embodiments, the symptoms of secondary hypogonadism comprise fatigue, lack of concentration, mood swings, decreased sexual desire, erectile dysfunction, infertility, hair loss, reduced muscle and bone mass, weight gain or a combination thereof.

Some embodiments are directed to a method of administering a hormone to a subject in need thereof, the method comprising administering the hormone via a needle-free injection to an injection site with a needle-free injection device.

In some embodiments, the needle-free injection device comprises a portable injector and a disposable syringe. In some embodiments, the disposable syringe is pre-loaded with a therapeutically effective amount of the hormone. In some embodiments, the hormone is testosterone. In some embodiments, the disposable syringe can be loaded with a variable amount of the hormone.

Some embodiments are directed to a method of hormone replacement therapy or the symptoms thereof where the method comprising administering to the subject a therapeutically effective amount of hormone by a needle-free injection.

In some embodiments, needle-free injection of a therapeutically effective amount of testosterone to the subject can be used in various forms of hormone replacement therapy. Clinicians have acknowledged that young transsexual adolescents suffer greatly due to the pubertal development. In order to address this dilemma, clinics have started treating young adolescents with puberty-suppressing medication if they fulfill eligibility and readiness criteria for gender reassignment. The delay in puberty allows children to avoid harmful hormone therapy before puberty where there is a high incidence of children with gender identity disorder that does not persist into adolescents. After this delay in puberty, males who wish to continue with puberty and females who wish to continue with gender reassignment must be given testosterone for induction of male puberty. Female-to-male transsexual persons must undergo hormone replacement therapy to achieve testosterone values in the normal male range (320-1,000 ng/dL). In some embodiments, female-to-male transsexual persons undergoing hormone replacement therapy follow similar treatment regimens as hypogonadal males. In some embodiments, hormone replacement therapy is required throughout the life of the person.

Some embodiments are directed to a method of minimizing fluctuations in testosterone levels in a subject diagnosed with hypogonadism, the method comprising serially administering to the subject a therapeutically effective amount of testosterone by needle-free injection to an injection site, wherein the subjects' testosterone levels are maintained within a range from between about 300 ng/dL and 900 ng/dL.

In some embodiments, minimizing fluctuations in testosterone levels in a subject diagnosed with hypogonadism comprises administering to the subject a pharmaceutical composition comprising a therapeutically effective amount of testosterone and a pharmaceutically acceptable carrier by needle-free injection. In some embodiments, the subject is refractory to testosterone therapy. In some embodiments, the increase in blood testosterone levels will be determined based on the age of the subject, or will be determined by a physician. In some embodiments, the subject is refractive to a topical testosterone therapy used to treat hypogonadism such as, but not limited to testosterone creams and gels. In some embodiments, the subject is refractory to an injectable testosterone therapy used to treat hypogonadism such as, but not limited to injectable pellets. In yet other embodiments the subject is refractory to all testosterone treatments used to treat hypogonadism.

In some embodiments, the subject is administered a therapeutically effective amount of testosterone by needle-free injection. In some embodiments, the subject is administered a therapeutically effective amount of testosterone by needle-free injection once every 12 hours. In some embodiments, the subject is administered a therapeutically effective amount of testosterone by needle-free injection once every 24 hours. In some embodiments, the subject is administered a therapeutically effective amount of testosterone by needle-free injection once every 36 hours. In some embodiments, the subject is administered a therapeutically effective amount of testosterone by needle-free injection once every 48 hours. In some embodiments, the subject is administered a therapeutically effective amount of testosterone by needle-free injection once every 60 hours. In some embodiments, the subject is administered a therapeutically effective amount of testosterone by needle-free injection once every 72 hours. In some embodiments, the subject is administered a therapeutically effective amount of testosterone by needle-free injection once every 168 hours. In some embodiments, the subject is administered a therapeutically effective amount of testosterone by needle-free injection once every 180 hours. In some embodiments, the subject is administered a therapeutically effective amount of testosterone by needle-free injection at any time between any of these values. In some embodiments, administering to the subject a therapeutically effective amount of testosterone by needle-free injection is performed on a weekly administration cycle wherein administering to the subject a therapeutically effective amount of testosterone by needle-free injection is performed three times a week on day 1 (0 hours), day 3 (48 hours), and day 5 (96 hours). In some embodiments, administering to the subject a therapeutically effective amount of testosterone by needle-free injection is performed on a weekly administration cycle wherein administration is performed at 0 hours, 48 hours and 96 hours after which no administration is given for 72 hours after which the cycle re-starts. For example, administering to the subject a therapeutically effective amount of testosterone by needle-free injection may be performed on a Monday, Wednesday, and Friday after the administration cycle restarts on the following Monday.

In some embodiments, administering to the subject a therapeutically effective amount of testosterone by needle-free injection at least once every 12 hours. In some embodiments, administering to the subject a therapeutically effective amount of testosterone by needle-free injection at least once every 24 hours. In some embodiments, administering to the subject a therapeutically effective amount of testosterone by needle-free injection at least once every 36 hours. In some embodiments, administering to the subject a therapeutically effective amount of testosterone by needle-free injection at least once every 48 hours. In some embodiments, the administering to the subject a therapeutically effective amount of testosterone by needle-free injection at least once every 60 hours. In some embodiments, administering to the subject a therapeutically effective amount of testosterone by needle-free injection at least once every 72 hours. In some embodiments, administering to the subject a therapeutically effective amount of testosterone by needle-free injection at least once every 168 hours. In some embodiments, administering to the subject a therapeutically effective amount of testosterone by needle-free injection at least once every 180 hours. In some embodiments, administering to the subject a therapeutically effective amount of testosterone by needle-free injection at any time between any of these values.

In some embodiments, administering to the subject a therapeutically effective amount of testosterone by needle-free injection delivers testosterone subcutaneously. In some embodiments, testosterone administration can be delivered subcutaneously to various areas of the body, including but not limited to the abdomen, chest, back, buttocks, face, neck, upper arms, lower arms, upper legs, lower legs, hands, feet, groin, pubic area, or external sexual organs. In other embodiments of the method, administering to the subject a therapeutically effective amount of testosterone by needle-free injection delivers testosterone intradermally. In some embodiments, testosterone administration can be delivered intradermally to various areas of the body, including but not limited to the abdomen, chest, back, buttocks, face, neck, upper arms, lower arms, upper legs, lower legs, hands, feet, groin, pubic area, or external sexual organs. In further embodiments of the method, administering to the subject a therapeutically effective amount of testosterone by needle-free injection delivers testosterone intramuscularly. In some embodiments, testosterone administration can be delivered intramuscularly to various areas of the body, including but not limited to the abdomen, chest, back, buttocks, face, neck, upper arms, lower arms, upper legs, lower legs, hands, feet, groin, pubic area, or external sexual organs. In certain embodiments of the method, the subject can be administered a therapeutically effective amount of testosterone by needle-free injection which is delivered by any other means of penetration of the skin or combinations thereof. In some embodiments, testosterone administration can be delivered to various areas of the body, including but not limited to the abdomen, chest, back, buttocks, face, neck, upper arms, lower arms, upper legs, lower legs, hands, feet, groin, pubic area, or external sexual organs.

In some embodiments, the subject self-administers the testosterone by needle-free injection. In some embodiments, the subject self-administers the testosterone by needle-free injection after training by a medical professional. In some embodiments, the subject will forego the necessity of clinical visits for each injection, offering reduced cost and time expenditures. In some embodiments, self-administration also provides the ability for the subject to administer the injection at any location. In some embodiments, the testosterone by needle-free injection is administered by a medical professional.

In some embodiments, a therapeutically effective amount of testosterone is about 1 mg to about 100 mg. In some embodiments, a therapeutically effective amount of testosterone is about 25 mg. In some embodiments, a therapeutically effective amount of testosterone is about 37.5 mg. In some embodiments, a therapeutically effective amount of testosterone is about 50 mg. In some embodiments, a therapeutically effective amount of testosterone is about 0.1 mg to about 150 mg, and a value between any of those values. In some embodiments, a therapeutically effective amount may be assessed in any manner known in the art, including but not limited to determining a subject as no longer hypogonadal. In some embodiments, a therapeutically effective amount can assessed by the amelioration of at least one symptom of hypogonadism. In some embodiments, symptoms of hypogonadism include, but are not limited to, fatigue, lack of concentration, mood swings, decreased sexual desire, erectile dysfunction, infertility, hair loss, reduced muscle and bone mass, weight gain or a combination thereof.

In some embodiments, the subjects testosterone levels are maintained within a range from about 400 ng/dL to about 900 ng/dL, from about 500 ng/dL to about 900 ng/dL, from about 600 ng/dL to about 900 ng/dL, from about 700 ng/dL to about 900 ng/dL, from about 800 ng/dL to about 900 ng/dL, or a range between any two of these values.

In some embodiments, serially administering to the subject a therapeutically effective amount of testosterone comprises one injection every 12 hours. In some embodiments, serially administering to the subject a therapeutically effective amount of testosterone comprises one injection every 24 hours. In some embodiments, serially administering to the subject a therapeutically effective amount of testosterone comprises one injection every 36 hours. In some embodiments, serially administering to the subject a therapeutically effective amount of testosterone comprises one injection every 48 hours. In some embodiments, serially administering to the subject a therapeutically effective amount of testosterone comprises one injection every 60 hours. In some embodiments, serially administering to the subject a therapeutically effective amount of testosterone comprises one injection every 72 hours. In some embodiments, serially administering to the subject a therapeutically effective amount of testosterone comprises one injection every 168 hours. In some embodiments, serially administering to the subject a therapeutically effective amount of testosterone comprises one injection every 180 hours. In some embodiments, administering to the subject a therapeutically effective amount of testosterone by needle-free injection is performed on a weekly administration cycle wherein administering to the subject a therapeutically effective amount of testosterone by needle-free injection is performed three times a week on day 1 (0 hours), day 3 (48 hours), and day 5 (96 hours). In some embodiments, administering to the subject a therapeutically effective amount of testosterone by needle-free injection is performed on a weekly administration cycle wherein administration is performed at 0 hours, 48 hours and 96 hours after which no administration is given for 72 hours after which the cycle re-starts. For example, administering to the subject a therapeutically effective amount of testosterone by needle-free injection may be performed on a Monday, Wednesday, and Friday after the administration cycle restarts on the following Monday.

In some embodiments, serially administering to the subject a therapeutically effective amount of testosterone by needle-free injection at least once every 12 hours. In some embodiments, serially administering to the subject a therapeutically effective amount of testosterone by needle-free injection at least once every 24 hours. In some embodiments, serially administering to the subject a therapeutically effective amount of testosterone by needle-free injection at least once every 36 hours. In some embodiments, serially administering to the subject a therapeutically effective amount of testosterone by needle-free injection at least once every 48 hours. In some embodiments, serially administering to the subject a therapeutically effective amount of testosterone by needle-free injection at least once every 60 hours. In some embodiments, serially administering to the subject a therapeutically effective amount of testosterone by needle-free injection at least once every 72 hours. In some embodiments, serially administering to the subject a therapeutically effective amount of testosterone by needle-free injection at least once every 168 hours. In some embodiments, serially administering to the subject a therapeutically effective amount of testosterone by needle-free injection at least once every 180 hours. In some embodiments, serially administering to the subject a therapeutically effective amount of testosterone by needle-free injection at any time between any of these values.

In some embodiments, the volume of testosterone administered by needle-free injection is from about 0.05 mL to about 1 mL, and any value in between these two values. In some embodiments, the volume of testosterone administered by needle-free injection is about 0.5 mL.

Embodiments herein are directed to methods of increasing testosterone levels in a subject in need thereof, the method comprising administering to the subject a therapeutically effective amount of testosterone by needle-free injection to an injection site. In some embodiments, administering to the subject a therapeutically effective amount of testosterone by needle-free injection comprises administering the testosterone using a needle-free injection device. In some embodiments, the needle-free injection device comprises ZetaJet delivery system. In some embodiments, the needle-free injection device is selected from a spring-powered injection device, a gas powered injection device and combinations thereof. In some embodiments, the injection site is the abdomen, chest, back, buttocks, face, neck, upper arms, lower arms, upper legs, lower legs, hands, feet, groin, pubic area, or external sexual organs. In some embodiments, the injection site is not directly over a blood vessel. In some embodiments, the subject is receiving testosterone through needle injection and wherein needle injection is substituted with needle-free injection. In some embodiments, administering to the subject a therapeutically effective amount of testosterone by needle-free injection is performed once every 48 hours with a 72 hour gap after three sequential administrations. In some embodiments, administering to the subject a therapeutically effective amount of testosterone by needle-free injection delivers testosterone subcutaneously. In some embodiments, administering to the subject a therapeutically effective amount of testosterone by needle-free injection delivers testosterone intradermally. In some embodiments, administering to the subject a therapeutically effective amount of testosterone by needle-free injection delivers testosterone intramuscularly. In some embodiments, the subject self-administers the testosterone by needle-free injection. In some embodiments, the testosterone by needle-free injection is administered by a medical professional. In some embodiments, a therapeutically effective amount of testosterone is from about 1 mg to about 100 mg. In some embodiments, a therapeutically effective amount of testosterone is about 50 mg. In some embodiments, the volume of testosterone administered by needle-free injection is from about 0.05 mL to about 1 mL. In some embodiments, the volume of testosterone administered by needle-free injection is from about 0.5 mL. In some embodiments, the testosterone levels of the subject 6 hours after administering to the subject a therapeutically effective amount of testosterone by needle-free injection are from about 300 ng/dL to about 900 ng/dL. In some embodiments, the subject is a human. In some embodiments, the subject is a human male. In some embodiments, the subject is a human male from about 40 to about 70 years of age. In some embodiments, the subject is clinically diagnosed with secondary hypogonadism. In some embodiments, the subject is a human male from about 40 to about 70 years of age clinically diagnosed with secondary hypogonadism. In some embodiments, the subjects serum total testosterone level is known. In some embodiments, the subject has a serum total testosterone level below about 300 ng/dL. In some embodiments, the subject has had a serum total testosterone level below about 300 ng/dL on at least two separate occasions prior to administration. In some embodiments, the subject is not taking a testosterone supplement, a testosterone pharmaceutical, a corticosteroid, a growth hormone supplement, dehydroepiandrosterone (DHEA), and luteinizing hormone-releasing hormone (LHRH) agonist or a combination thereof. In some embodiments, the subject is not pregnant. In some embodiments, the subject does not have a history of prostate cancer, a current diagnosis of prostate cancer or a combination thereof. In some embodiments, the subject has had a prior Testopel insertion and wherein at least one testosterone level in the L range and 5 months have passed since insertion. In some embodiments, administering to the subject a therapeutically effective amount of testosterone by needle-free injection causes less pain to the subject than administration of testosterone by needle injection. In some embodiments, administering to the subject a therapeutically effective amount of testosterone by needle-free injection is more tolerable to the subject than administration of testosterone by needle injection. In some embodiments, administering to the subject a therapeutically effective amount of testosterone by needle-free injection results in less post injection wetness than administering testosterone by needle injection. In some embodiments, administering to the subject a therapeutically effective amount of testosterone by needle-free injection results in less redness, bruising, induration, or a combination thereof than administering testosterone by needle injection. In some embodiments, administering to the subject a therapeutically effective amount of testosterone by needle-free injection results in greater dispersion than administration with a needle injection. In some embodiments, administering to the subject a therapeutically effective amount of testosterone by needle-free injection does not cause damage to skin cells at the site of injection.

### EXAMPLES

The following example is offered to illustrate, but not to limit the claimed invention.

### Example 1

### Use of a Novel Subcutaneous Needle-Free Technique to Deliver Testosterone in Hypogonadal Men

The goal of the study is to determine if a needle-free injector can consistently deliver testosterone in a daily delivery, safely and effectively.

Fourteen men ages (40-70) will be enrolled in the study for SC daily injections of 25 mg of testosterone cypionate. Dosage was calculated based on new formulations of daily Androgel® with one pump (approximately 20 mg). Cream dosage of 40-80 mg a day is typical. Injection rates are almost twice as bioavailable as creams, therefore safe, reasonable levels of testosterone of 25 mg a day was chosen. The study will enroll the first four subjects and after two weeks ensure that testosterone levels are not above 900 ng/dL for any of these patients, or less than 300 ng/dL, for any of these subjects.

To confirm appropriate dosing for the study, the initial four subjects of the study will be followed for two weeks to evaluate testosterone levels before accruing further subjects into the study. Each of these four subjects' levels will be evaluated and at the second week into the study, their respective testosterone levels will be classified as either, Low (L), Normal (N), or High (H), depending on their levels in relation to specified range 300-900 ng/dL. The remaining projection of the study will depend on the ratios of these four. Table 1 below will determine the projection.

TABLE 1
Treatment Projections

| | | | |
|---|---|---|---|
| L | L | L | The original 25 mg dose for the remainder of the study will |
| L | L | L | be increased by 50% to 37.5 mg of T/day; at two week |
| L | L | L | blood draw all patients will be switched to every other day |
| L | N | H | (QOD) dosing if T levels are H |
| N | N | N | Study is continued at 25 mg/day with no option for every |
| N | N | N | other day (QOD) dosing at two weeks if levels are H |
| N | N | L | |
| N | L | L | |
| N | N | H | Study is continued at 25 mg/day WITH option for every |
| N | N | H | other day (QOD) dosing at two weeks if levels are H |
| N | H | L | |
| H | H | L | |
| H | H | H | The 25 mg dose will remain the same, however all |
| H | H | H | participants will be switched to every other day (QOD) |
| H | H | H | dosing |
| H | N | L | |

For all subjects, a safety precaution will be instituted into the study, if on day 14 the total testosterone level is greater than 1000 ng/dL: same dose of testosterone will be given but the frequency will be changed to every other day.

TABLE 2
Study Data to be collected to Achieve Objectives

| | |
|---|---|
| Objective #1 | Blood levels for Testosterone as indicated below |
| Objective #2 | Subjects' immediate reports of pain at the injection site |
| Objective #3 | Evaluation of the injection site reaction |
| Objective #4 | Subjects' responses to questions on acceptability of the |
| | Zetajet |
| Objective #5 | Aging Male's Symptom (AMS) before and after treatment |

TABLE 3
Hypothesis testing

| | |
|---|---|
| Hypothesis #1 | Daily Subcutaneous injections of Testosterone using the |
| | Zetajet provide adequate blood levels |
| Hypothesis #2 | Needle-free injections are more acceptable than needle |
| | injections |
| Hypothesis #3 | Needle-free provides a better alternative to intramuscular |
| | weekly or monthly injections |
| Hypothesis #4 | Subject's response on AMS reflects improved quality of |
| | life after treatment |

Study Population

General: 14 total patients age 40-70, males with clinically diagnosed secondary hypogonadism. Subject Age/Gender: The gender and ages of the subjects will reflect that of a normal distribution for the general study population.

Concomitant Drugs: Subjects cannot be taking any other testosterone supplements/pharmaceuticals, corticosteroids, growth hormone supplement, DHEA, or LHRH agonist.

Subject Selection

Admission Procedures: Volunteers willing to participate and grant informed consent.

Screening Process: The clinical monitors will screen subjects per the following inclusion and exclusion criteria and the previously listed study population specifics. Urine pregnancy test will be administered to females at screening.

Inclusion/Exclusion Criteria

Inclusion Criteria: Healthy normal volunteers between 18 and 55 years old willing to participate and grant informed consent. Each subject must have documented at least two serum total testosterone levels that were below 300 ng/dL. Subjects cannot be currently taking any other testosterone supplements or medication. They must have had a one-month “washout” prior from intramuscular injections or any dermal therapy. If he had prior Testopel insertions, one testosterone level in the L range and 5 months prior to insertion must have past for inclusion. Patients with a prior history of prostate cancer may not be included in the study.

Exclusion Criteria: Criteria for exclusion include a current or prior diagnosis of prostate cancer; carry an acute illness, and short- or long-term drug therapy.

Informed Consent: All participants will sign a consent form that has been approved by TBD Institutional Review Board (IRB).

Equipment

Bioject Inc. will provide Zetajet devices to the study site. The study site will provide antiseptic prep wipes, Band-Aids, gloves, and Biohazard waste containers as required. Bioject, Inc. will supply Case Report Forms (CRFs).

Conduct of Study

Study Period/Procedures

Period: 30 days

Procedure:

Step 1. Subjects will be provided with a consent form, which they will sign if they review the form thoroughly and understand completely their role as subjects in the study.

Step 2. Subjects will be trained by a nurse on the use of the Zetajet device. Each patient will give 1 injection of normal saline as a practice injection.

Step 3. Subjects will receive a subcutaneous injection of 25 mg of testosterone cypionate (0.5 mL) in the abdominal region using the ZetaJet. Each subject will be taught how to draw up the solution and administer the medication daily at the same time of day.

Step 4. 10 minutes after the first injection, reports of injection pain will be elicited from subjects and any site reactions will be recorded. Subjects will be asked to assess the injections with regard to tolerability.

Subjects will return to the clinic in 24 hours for a second injection and the same assessment will be made as the first injection assessment, then the following injections will be performed at home.

TABLE 4
Summary: (In office)

| | |
|---|---|
| 1st injection | Office Nurse to administer practice injection with normal |
| | saline |
| 2nd injection | Subject to self-inject with normal saline |
| 3rd injection | Subject to self-inject with medication |
| 4th injection | Subject to return to office and self-inject with medication |
| | with nurse observing (Thereafter, self-inject every day at |
| | home) |

Study Time-line

| | | | | Hospital Free and |
|---|---|---|---|---|
| | Testosterone | Skin | AMS | total T/Estradiol |
| | level | examination | score | level |
| Day 1 early | X | X | X | X |
| Day 1 late | X | X | | |
| (6 hrs post) | | | | |
| Day 2 early | X | X | | |
| Day 7 | X | X | | |
| Day 14 | X | X | | |
| Day 30 | X | X | X | X |

Safety Parameters

Introduction: Qualified health care professionals will administer the injections, elicit subjective responses, and evaluate the injection site for 30 minutes afterwards.

Adverse Events (Reactions and Complications): For unanticipated complications, treatment is at the discretion of the investigator. The study may immediately cease until the investigators can make an assessment. A severe adverse event may cause subject entry to be postponed until the adverse event is thoroughly investigated and resumption of the study is deemed appropriate.

Warnings and Precautions: Skin at the injection site should be intact and free from irritation, bruises, and abrasions. Do not use areas that are sensitive to touch or rough.

The Zetajet should not be positioned directly over a blood vessel when administering an injection. Although intravascular injection with the Zetajet is unlikely, injection over a vessel may cause adverse reactions including perforation, bruising, swelling, or tenderness.

Device Failures/Replacements: In the event where a device failure occurs, it will be documented with a description of all events leading up to and including the failure.

Patient Complaints: All subject complaints will be recorded on the Case Report Forms.

Subject Withdrawal

Subjects will be removed from the study at the request of the subject, if the subject does not conform to inclusion criteria, in the event of a significant departure from protocol, or in the event of a severe adverse reaction. The investigators reserve the right to remove subjects from the study at their discretion.

Statistical Considerations and Methods

Statistical Plan: Patients are served as their own control. Prior baseline testosterones will be averaged and the study levels will be analyzed by one-way t-test averages to test for statistical significant change above baseline. AMS scores will be evaluated and statistical comparisons of before and after treatment will be made.

Criteria for Exclusion of Data from Analysis: Patient outliers will be analyzed for the cause(s) of the anomalous results and will be treated appropriately. Device failures will be treated as described above and the corresponding data points will be eliminated from tabulation of the results.

Safety Data Analysis (If applicable): All adverse events will be listed and described on the Case Report Forms (CRFs).

Quality Assurance Procedure

The Case Report Forms (CRFs) will be verified by the clinical monitors for accuracy. Data analysis will be reviewed and confirmed by the investigators prior to pursuing formal publication.

### Example 2

### Use of a Novel Subcutaneous Needle-Free Technique to Deliver Testosterone in Hypogonadal Men—Clinical Study Report

The goal of the study is to determine if a needle-free injector can consistently deliver frequent administrations (daily or three times weekly) of testosterone in a safe and effective manner. To answer this specific question, the study was designed to determine the efficacy (defined as testosterone serum levels, wetness, pain, and clinical questionnaire) of the combined product (cross labeled) of needle-free injection of testosterone cypionate hypogonadal men. This includes both objective (testosterone free and total levels) and subjective (pain and clinical questionnaire).

Fourteen men ages 40-75 were enrolled in a prospective, non-blinded study that presented to a general urology practice with a primary clinical complaints of symptoms attributed to primary hypogonadism and who demonstrated at least two prior testosterone levels below 300 ng/dl. Each subject was given thorough instruction on performing self needle-free injections with the Zetaj et. Once each subject demonstrated successful use of the device using saline self-injection, subjects began self-injection with 0.25 cc (25 mg) of testosterone cypionate daily in the abdomen rotating sites. Pharmacokinetic evaluation using testosterone levels were obtained at specified intervals. Secondary endpoints, both objective and subjective, included E2 levels, pain score diaries, and AMS scores obtained before and after one month of treatment. A dose confirmation strategy was conducted to achieve likelihood of valid dose.

TABLE 6
Study Time-line
Study Time-line

| | | | | Hospital Free and |
|---|---|---|---|---|
| | Testosterone | Skin | AMS | total T/Estradiol |
| | level | examination | score | level |
| Day 1 early | X | X | X | X |
| Day 1 late | X | X | | |
| (6 hrs post) | | | | |
| Day 2 early | X | X | | |
| Day 7 | X | X | | |
| Day 14 | X | X | | |
| Day 30 | X | X | X | X |
| Day 60 | X | X | | |
| Day 120 | X | X | | |

Changes in the Conduct of the Study

On Jan. 26, 2013, IRBS approved a modification of the CP-445 study. Due to a clear efficacy and patient acceptance at day 30, an extension of the study was requested to follow the fourteen subject's testosterone levels at a less frequent dosing interval and increased dosage. There was no deviation to the protocol as originally designed for day 0-30. Nor was there any deviation to the conduct of the study to day 120. This modification allowed following subjects out to day 120 on a Monday, Wednesday, Friday dosing of 50 mg of testosterone cypionate. Only T levels at day 60 and 120 were assessed.

Demographics

The average age of the fourteen men was 61 (42-73) with an average BMI of 30.8. Men had an average starting testosterone level 184 ng/dl (82-287). Aging Male Score evaluating the severity of hypogonadal symptoms was demonstrated an average of 50 indicating the population studied had clinically significant alterations in sexual and lifestyle domains criteria consistent with hypogonadism.

Initial Post Injection Findings

Five Minute Visual/Verbal Analog Pain Scale

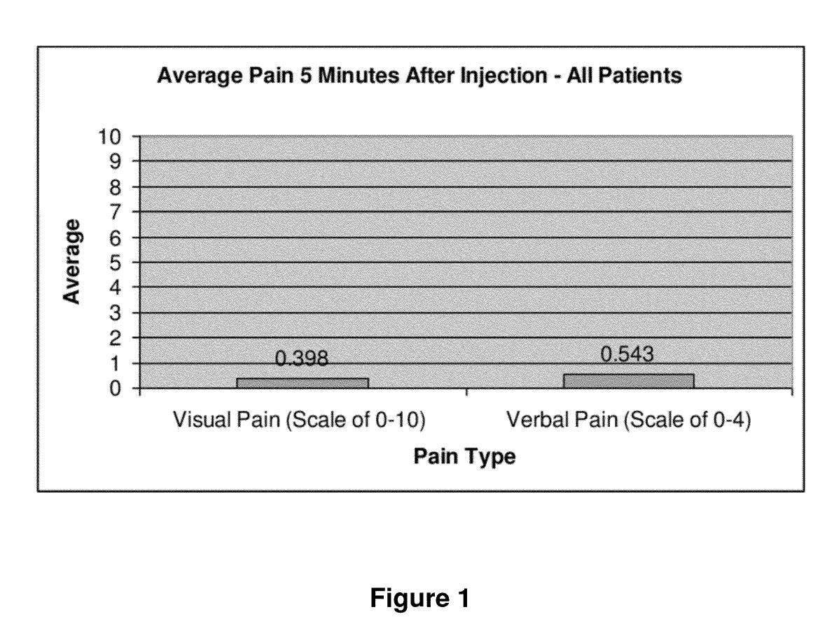

Testosterone levels using daily injection had little to no pain associated with the injections. Men tolerated the injections with minimal discomfort. As can be seen in FIG. 1, five minutes after the injection, men demonstrated a very low visual analog pain score of 0.4 (0-10). Verbal analog pain score as well demonstrated a low description of discomfort soon after the injection with an average score of 0.54 (0-4).

Thirty Minute Visual Analog Pain Scale

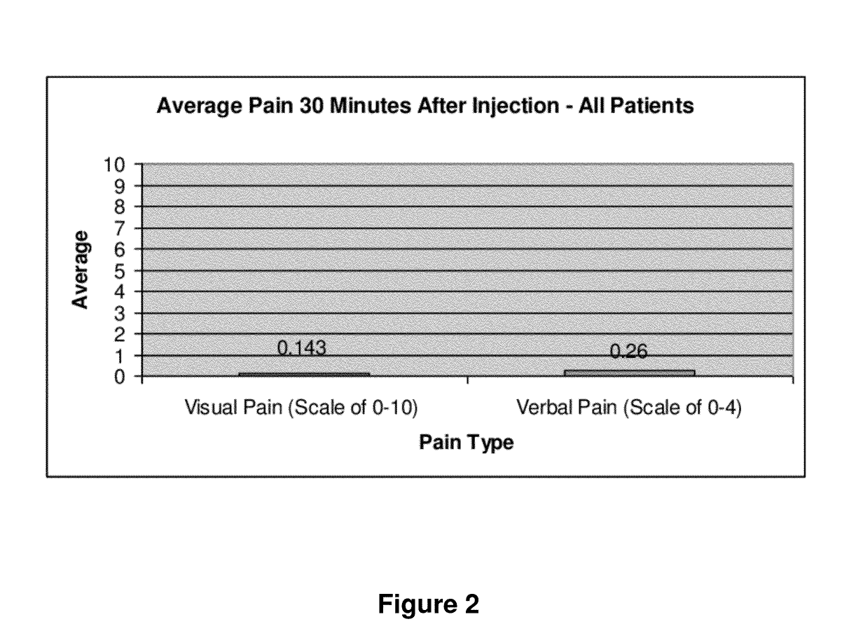

As can be seen in FIG. 2, no delayed pain response was found after the injection with Visual/Verbal scores of 0.14/0.26, respectively with a visual pain scale of 0-10, with 10 being the most painful and a verbal pain scale of 0-4 with 4 being the most painful. Three subjects noted at day 60 patient interview and coordinator visit for testosterone assay that increased “stinging” and subdermal knots that remained for two days when the 200 mg/ml testosterone cypionate in sesame seed oil vehicle was injected. From day 60-90, all subjects were verified to have cottonseed vehicle in the commercially available formulation via Watson Pharmaceutical.

Testosterone Levels

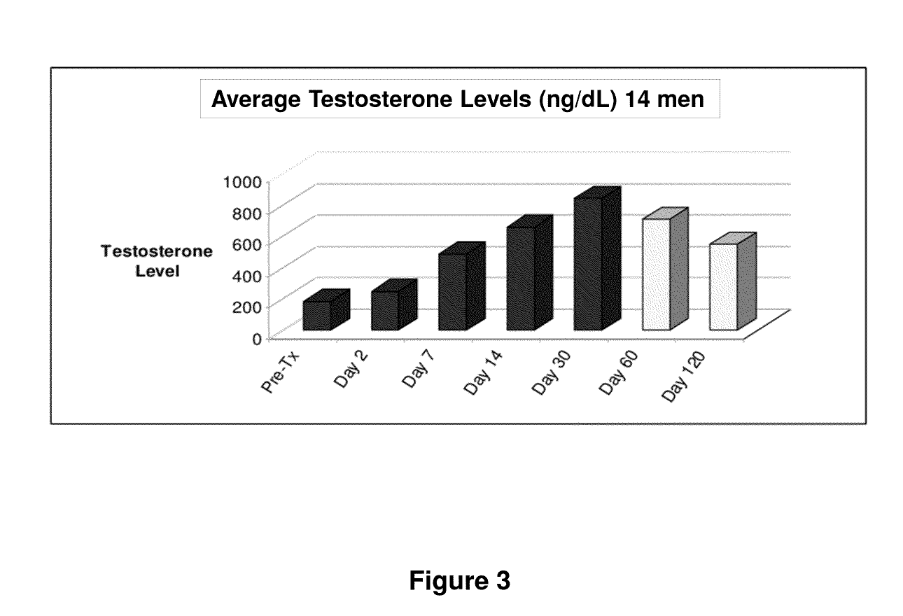

FIG. 3 depicts average testosterone levels over the course of the study. Free and total testosterone levels consistently rose from 6 hours to day 30 at the 25 mg daily Zetajet injection. Four men had levels higher than the normal range (supraphysiologic) above 975.0 ng/dl, with an average of 1203 ng/dl. Three of these subjects continued with the QODWO (Every other day dosing with weekend off) regimen and each of these men had over the extended course of the study corrected to the normal range with an average of 519 ng/dl. Free testosterone levels also had over four fold increase from an average 8.76 ng/dl to 37.5 ng/dl.

AMS Score

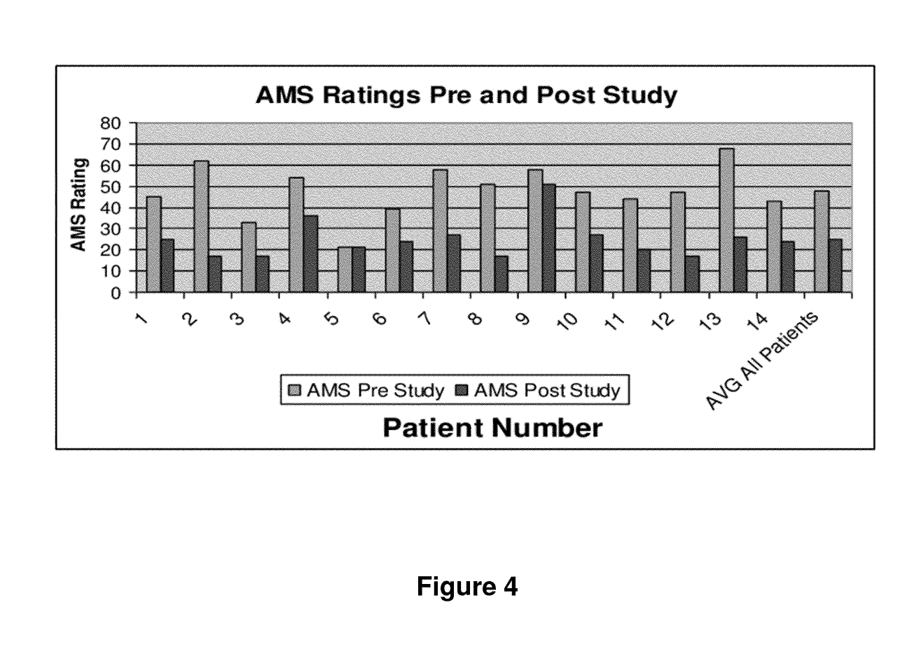

Thirteen of fourteen completed the Aging Male Score (AMS) questionnaire (Table 7) as designed with a decrease from an average of 50 points to an average of 25 resulting in an average of 50% reduction in bothersome hypogonadal symptoms as can be seen in FIG. 4. Only one patient did not see a noted decrease in AMS during the study though there was a change in the noted symptoms. AMS scoring was based on a rating of symptom severity ranging from no symptoms with the lowest score of 1 to severe symptoms with a score of 6.

TABLE 7
Aging Male Score (AMS) Questionnaire
Which of the following symptoms apply to you at this time? Please mark the appropriate
box for each symptom. For symptoms that do not apply, please mark “none”.

| | Symptoms: | | | | |
|---|---|---|---|---|---|
| | | | | | Extremely |
| | None | Mild | Moderate | Severe | severe |
| Score | 1 | 2 | 3 | 4 | 5 |
| 1. Decline in your feeling of general | □ | □ | □ | □ | □ |
| wellbeing (general state of health, subjective | | | | | |
| feeling) | | | | | |
| 2. Joint pain and muscular ache (lower back | □ | □ | □ | □ | □ |
| pain, joint pain, pain in a limb, general back | | | | | |
| pain) | | | | | |
| 3. Excessive sweating (unexpected/sudden | □ | □ | □ | □ | □ |
| episodes of sweating, hot flushes | | | | | |
| independent of strain) | | | | | |
| 4. Sleep problems (difficulty in falling | □ | □ | □ | □ | □ |
| asleep, difficulty in sleeping through, waking | | | | | |
| up early and feeling tired, poor sleep, | | | | | |
| sleeplessness) | | | | | |
| 5. Increased need for sleep, often feeling | □ | □ | □ | □ | □ |
| tired | | | | | |
| 6. Irritability (feeling aggressive, easily | □ | □ | □ | □ | □ |
| upset about little things, moody) | | | | | |
| 7. Nervousness (inner tension, restlessness, | □ | □ | □ | □ | □ |
| feeling fidgety) | | | | | |
| 8. Anxiety (feeling panicky) | □ | □ | □ | □ | □ |
| 9. Physical exhaustion/lacking vitality | □ | □ | □ | □ | □ |
| (general decrease in performance, reduced | | | | | |
| activity, lacking interest in leisure activities, | | | | | |
| feeling of getting less done, or achieving | | | | | |
| less, of having to force oneself to undertake | | | | | |
| activities) | | | | | |
| 10. Decrease in muscular strength (feeling of | □ | □ | □ | □ | □ |
| weakness) | | | | | |
| 11. Depressive mood (feeling down, sad, on | □ | □ | □ | □ | □ |
| the verge of tears, lack of drive, mood | | | | | |
| swings, feeling nothing is of any use) | | | | | |
| 12. Feeling that you have passed your peak | □ | □ | □ | □ | □ |
| 13. Feeling burnt out, having hit rock-bottom | □ | □ | □ | □ | □ |
| 14. Decrease in beard growth | □ | □ | □ | □ | □ |
| 15. Decrease in ability/frequency to perform | □ | □ | □ | □ | □ |
| sexually | | | | | |
| 16. Decrease in the number of morning | □ | □ | □ | □ | □ |
| erections | | | | | |
| 17. Decrease in sexual desire/libido (lacking | □ | □ | □ | □ | □ |
| pleasure in sex, lacking desire for sexual | | | | | |
| intercourse) | | | | | |
| Have you got any other major symptoms? | Yes | □ | No | □ | □ |
| If yes, please describe: | | | | | |

Adverse Events

No severe adverse events were noted in the entire study. No allergic response or major bruising noted in either concentrations of commercially available testosterone cypionate (100 mg/ml vs. 200 mg/ml formulations).

Site Reactions

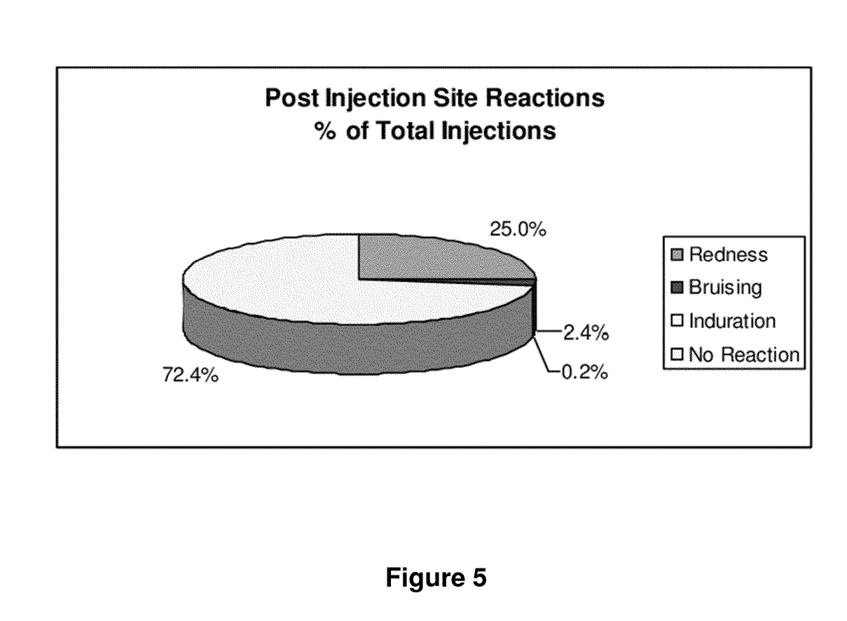

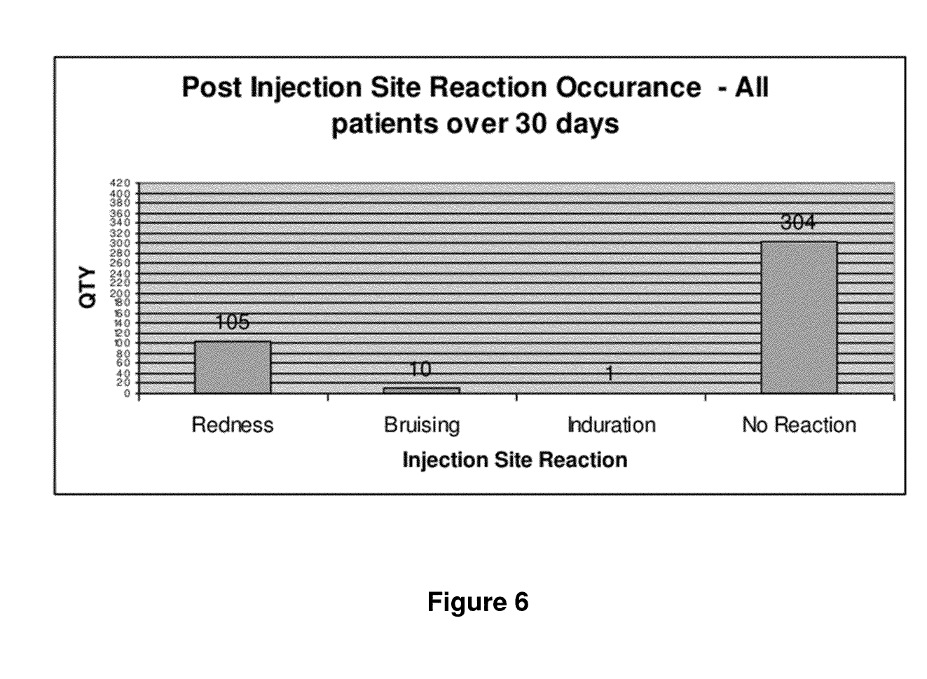

While the majority of injections yielded no adverse reactions, a quarter of the injections given resulted in minor redness and bruising. Of the 14 patients 9 recorded symptoms of redness, 5 symptoms of bruising and one an induration. None of these reactions required medical follow up or dissuaded any patient from discontinuing treatment. See section on Preference for additional data on patient preference and comparison of needle-Free injection methods to needle and syringe. FIG. 5 depicts post injection site reactions as a percentage of the total number of injections for 14 patients over a 30 day period. FIG. 6 depicts post injection reaction occurrence for all patients over a thirty day period and shows occurrences for 14 patients over 30 days and the total number of injections administered.

Wetness

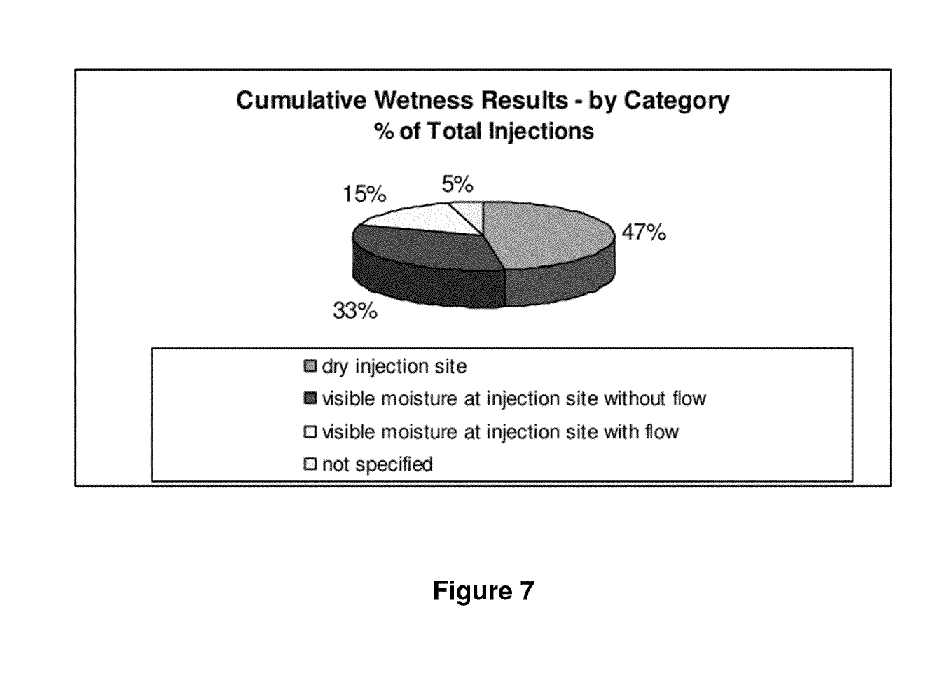

Wetness results show roughly half of the injections were dry with no visible wetness on the surface of the skin. Of the wet injections noted the majority of them did not show visible flow of medication. Wetness at the injection site is believed to be attributed to moisture on the tip of the syringe prior to the injection per recommended technique. Wetness with visible moisture flow at the site of the injection is thought to result from improper injection technique and a lack of fluid pressure in the nozzle because of the technique. Results for wetness varied across all patients. FIG. 7 depicts cumulative wetness results by category as a percentage of total injections.

Preference

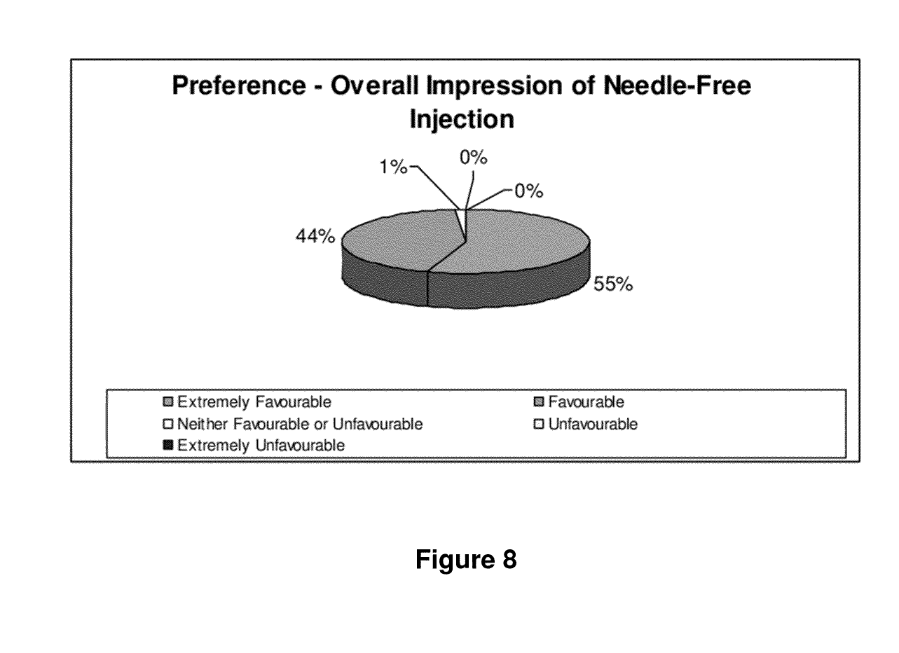

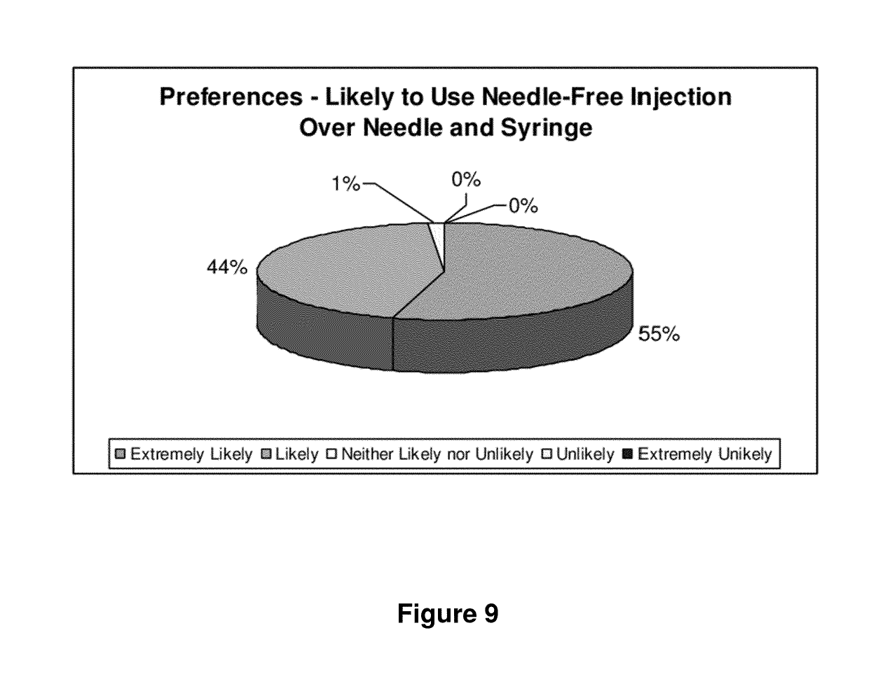

Overall impression of the needle-free delivery method was quite high in this study and patients were consistently positive in favor of needle-free injection over needle and syringe when given the option. There were no instances of patients reporting an unfavorable impression or recording that they would not prefer to use needle-free over needle and syringe. All subjects were very satisfied with the mode of treatment. Of the subjects who concluded the 120 day study extension all have requested to continue with needle-free injections over other conventional treatments/therapies. FIG. 8 depicts the overall impression of needle-free injection. FIG. 9 depicts the patients' likelihood of using needle-free injection over needle and syringe.

Clinical Coordinator/Registered Nurse Summary of “Patient Interviews”, Pre and Post treatment Prior to the start of the case study, each participant was screened by the physician and deemed to meet the criteria that the study was based upon. They each previously had two serum testosterone levels that revealed hypogonadism, as well as a variety of reasons for seeking alternative testosterone treatment. The reasons verbalized (in no order of importance) are as follows:
Displeasure of symptoms of testosterone level “swinging up and down;”
Fear of topically applied testosterone causing harm to wives, daughters and granddaughters;
Painful intramuscular and surgical interventions for testosterone replacement;
Lack of spontaneity in their “sexual life;”
Difficulty scheduling office visit for intramuscular injections;
Lack of sexual confidence;
Insomnia, fatigue, and mood swings;
Financial limitation
Various time and scheduling conflicts.

The initial meeting began, with each participant receiving an explanation of the nature and purpose of the study, information regarding their rights pertaining to the case study and obtaining consent from each participate.

During day zero of the study, each participant was instructed on proper administration of medication, syringes, dose measurements and utilization of the Zetajet Needle-Free Device through verbal and visual teaching aids. The nurse provided a demonstration of proper injection technique using the Zetajet Device and sterile water. The participant then had an opportunity to repeat this process for self-injection in order to demonstrate/prove their ability to perform self-injections using the Zetajet Device.

By day seven of the study, thirteen out of fourteen participants demonstrated ability and verbalized confidence with self-administration using the Zetajet Device. These participants required minimal assistance that could be provided remotely over the phone. Only one participant was unable to “master self-injections using the Zetajet.” This particular participant was provided with additional instructions on several occasions; however, it later became apparent this individual was in the process of losing cognition/memory unrelated to the study. This participant was advised to consult his primary care physician.

Each participant completed the Aging Male Symptoms Score (AMS) questionnaire at the beginning as well as the end of the case study. The results of the questionnaire showed that all participants saw an improvement in their AMS score at the completion of the study with the exception of one patient who saw a change in symptoms but who maintained the same score at the end of the treatment period.

Participants were notably eager to experience a “new” technique for testosterone replacement. They all verbalized an improved sense of well-being. They had numerous questions about the availability of the Zetajet Device because of its needle-free aspect and the convenience that comes with the ability to self-inject. At the completion of the study, one major concern from participants was that they would have to return to their previous means of testosterone replacement therapy and the clinic received several concerned calls from patients with questions/concerns regarding the return of their Bioject Zetajet Devices.

Case Study Participates Comments

TT (firefighter) reported that several of his friends have to visit the doctor's office biweekly. He feels lucky that he can self-administer and not feel the “ups and downs.” He did not experience any problems using the Zetaj et.

GG (contractor) reported that he will use the website to order supplies. He verbalized confidence in using the Zetajet stating that there was “No pain unless I rush and don't do it right.” His wife is happy and he no longer worries his daughters will come in contact with the topical testosterone cream he was using. ED is better. He wants to continue using the Zetajet for his therapy.

SO (engineer) no problems noted.

MS (retiree) laughing joked that his young wife thanks me. I can travel, have better energy and not worry about appointments. This works for me. No pain. He plans to order supplies for continued use.

GW (salesman) reported that he had more energy and was sleeping better. He was glad he got to keep the device and hopes his brother gets in one of these studies. He will be ordering additional supplies so he can continue to use the Zetajet Device.

RS (grandfather) voiced that he has been taking the testosterone using the Zetajet. The “shots are doing great and I have a huge amount of energy.” He stated that he is loving life and dating.

DH (golfer) likes the Zetajet. I don't have erections or sex as often as I wish, but then I'm not as young as I used to be either. “Some is a lot better than none.”

HC (factory worker) treatment with the Zetajet was good and he has no complaints.

DL (real estate) I can manage my own injection here in clinic. When I get home its different. Dr Marotte is now seeing me in the office.

FS (grocery stocker) I am doing very well. The pharmacist was able to get my medicine covered on my insurance. He keeps giving me needles. I don't need them!

Discussion

Needle-free injection, whether daily or QODWO dosing, effectively treated all men in the study using commercially available testosterone cypionate. The study's primary endpoint of testosterone level demonstrated normal levels in 12/14 (85%) men by one week, 13/14 men at two weeks (86%) and 14/14 (100%) men at day 30.

Daily dosing regimen of 25 mg a day was well tolerated, improved symptoms and resulted in over four fold increase in average testosterone levels from baseline. Injections with QODWO dosing at 50 mg demonstrated a 3 fold increase in testosterone levels generating normal levels in 11 out of 11 men who completed the 120 day study extension. In comparing daily vs. QODWO dosing, the later had an average of 35% lower total testosterone levels than daily injections, however no men had supraphysiologic levels of testosterone as four subjects demonstrated with daily dosing.

While wetness results were varied a minority of men had visible “flow” of moisture after injection. Men had a clinical improvement by 50% of the AMS score demonstrating improvement in hypogonadal symptoms. An interesting observation was made when testosterone cypionate in sesame seed oil was used: larger red “wheel” at the injection site, a firm “knot” in the subcutaneous tissues beneath the site that remained for 2-3 days, as lastly, a lingering “stinging” pain after the injection. This proof of concept study used a concentration of 100 mg/ml manufactured by Pfizer, Inc. This commercially available testosterone cypionate in cottonseed oil was injected 0.25 cc of solution daily. The study was extended to every other day dosing with weekends off after thirty days of successful testosterone levels in all 14 patients studied. A switch was made in concentration of testosterone cypionate in order to keep the same amount of solution injected of 0.25 cc, therefore a 200 mg/ml solution of testosterone cypionate was purchased from wholesaler distributor Medex Care, Inc. We used testosterone cypionate 250 mg per ml in sesame seed oil for the beginning of the extended study. Early in the every other day dosing, many of the men complained of more “stinging” sensation and three patients developed subcutaneous knots and more erythematous skin reactions. Once recognized that the only different non-active drug components was the sesame seed oil, the men were placed on Watson testosterone cypionate 200 mg/ml in cottonseed oil. Patients then had no further stinging complaints.

In future studies, strict adherence to one manufacturer, Paddock Labs pharmaceuticals 200 mg/ml of cottonseed in single dose 1 ml vials will be used. Patients will take 0.25 mg per dose or 50 mg every Monday, Wednesday, and Friday. Adverse event monitoring for any skin or subdermal knots forming will be recorded from Case Report Forms and a detail clinical evaluation during the testosterone blood draws.

Conclusion

Needle-free injection with testosterone cypionate in cottonseed oil was a safe, well tolerated, and effective mode of treatment in treating 14 hypogonadal men without resulting additional pain post injection. All subjects were very satisfied with the mode of treatment. Of the fourteen subjects who concluded the 120 day study extension eleven have requested to continue with needle-free injections over other conventional treatments/therapies. Clear patient preference and successful self-administration makes this needle-free injection treatment method viable for future consideration.

Future Direction

Results from this study demonstrate that the tolerability and T levels were optimized at the 50 mg QODWO dosing by preventing supraphysiologic levels as seen in daily injections in 4 of the 14 men at day 30. A second prospective trial will be designed to study 10 subjects T level pharmacokinetics from early in QODWO dosing and to measure AMS questionnaire at day 30 and 120. Testosterone cypionate exclusively in cottonseed oil will be used to avoid any dermal reactions or increased pain response. The American Endocrine Society has compared modalities of delivery of T treatments and has listed advantages and disadvantages for these therapies specifically looking at tolerability, safety, and hormonal effects with respect to dyhydroxytestosterone levels (DHT) and estradiol levels. This second study will examine these levels and compare to conventional weekly intramuscular injections, creams/gels, patches, and pellet implants.

What is claimed:

1. A method of increasing testosterone levels in a subject in need thereof, the method comprising administering to the subject a therapeutically effective amount of testosterone by needle-free injection to an injection site.

2. The method of claim 1, wherein administering to the subject a therapeutically effective amount of testosterone by needle-free injection comprises administering the testosterone using a needle-free injection device.

3. The method of claim 2, wherein the needle-free injection device comprises the ZetaJet delivery system.

4. The method of claim 2, wherein the needle-free injection device is selected from a spring-powered injection device, a gas-powered injection device and combinations thereof.

5. The method of claim 1, wherein the injection site is the abdomen, chest, back, buttocks, face, neck, upper arms, lower arms, upper legs, lower legs, hands, feet, groin, pubic area, or external sexual organs of the subject.

6. The method of claim 1, wherein the injection site is not directly over a blood vessel.

7. The method of claim 1, wherein the subject is receiving testosterone through needle injection and wherein needle injection is substituted with needle-free injection.

8. The method of claim 1, wherein the subject is refractory to testosterone therapy.

9. The method of claim 1, wherein administering to the subject a therapeutically effective amount of testosterone by needle-free injection is performed at least once every 168 hours.

10. The method of claim 1, wherein administering to the subject a therapeutically effective amount of testosterone by needle-free injection is performed once every 24 hours.

11. The method of claim 1, wherein administering to the subject a therapeutically effective amount of testosterone by needle-free injection is performed once every 48 hours.

12. The method of claim 1, wherein administering to the subject a therapeutically effective amount of testosterone by needle-free injection is performed once every 48 hours with a 72 hour gap after three sequential administrations.

13. The method of claim 1, wherein administering to the subject a therapeutically effective amount of testosterone by needle-free injection delivers testosterone subcutaneously.

14. The method of claim 1, wherein administering to the subject a therapeutically effective amount of testosterone by needle-free injection delivers testosterone intradermally.

15. The method of claim 1, wherein administering to the subject a therapeutically effective amount of testosterone by needle-free injection delivers testosterone intramuscularly.

16. The method of claim 1, wherein the subject self-administers the testosterone by needle-free injection.

17. The method of claim 1, the testosterone by needle-free injection is administered by a medical professional.

18. The method of claim 1, wherein a therapeutically effective amount of testosterone is from about 1 mg to about 100 mg.

19. The method of claim 1 wherein a therapeutically effective amount of testosterone is about 25 mg.

20. The method of claim 1 wherein a therapeutically effective amount of testosterone is about 37.5 mg.

21. The method of claim 1 wherein a therapeutically effective amount of testosterone is about 50 mg.

22. The method of claim 1, wherein the volume of testosterone administered by needle-free injection is from about 0.05 mL to about 1 mL.

23. The method of claim 1, wherein the volume of testosterone administered by needle-free injection is about 0.5 mL.

24. The method of claim 1, wherein testosterone levels of the subject 6 hours after administering to the subject a therapeutically effective amount of testosterone by needle-free injection are from about 300 ng/dL to about 900 ng/dL.

25. The method of claim 1, wherein the subject is a human.

26. The method of claim 1, wherein the subject is a human male.

27. The method of claim 1, wherein the subject is a human male from about 40 to about 70 years of age.

28. The method of claim 1, wherein the subject is clinically diagnosed with secondary hypogonadism.

29. The method of claim 1, wherein the subject is a human male from about 40 to about 70 years of age clinically diagnosed with secondary hypogonadism.

30. The method of claim 1, wherein the subjects serum total testosterone level is known.

31. The method of claim 1, wherein the subject has a serum total testosterone level below about 300 ng/dL.

32. The method of claim 1, wherein the subject has had a serum total testosterone level below about 300 ng/dL on at least two separate occasions prior to administration.

33. The method of claim 1, wherein the subject is not taking a testosterone supplement, a testosterone pharmaceutical, a corticosteroid, a growth hormone supplement, DHEA, and LHRH agonist or a combination thereof.

34. The method of claim 1, wherein the subject is not pregnant.

35. The method of claim 1, wherein the subject does not have a history of prostate cancer, a current diagnosis of prostate cancer or a combination thereof.

36. The method of claim 1, wherein the subject has had a prior Testopel insertion and wherein at least one testosterone level in the L range and 5 months have passed since insertion.

37. The method of claim 1, wherein administering to the subject a therapeutically effective amount of testosterone by needle-free injection causes less pain to the subject than administration of testosterone by needle injection.

38. The method of claim 1, wherein administering to the subject a therapeutically effective amount of testosterone by needle-free injection is more tolerable to the subject than administration of testosterone by needle injection.

39. The method of claim 1, wherein administering to the subject a therapeutically effective amount of testosterone by needle-free injection results in less post injection wetness than administering testosterone by needle injection.

40. The method of claim 1, wherein administering to the subject a therapeutically effective amount of testosterone by needle-free injection results in less redness, bruising, induration, or a combination thereof than administering testosterone by needle injection.

41. The method of claim 1, wherein administering to the subject a therapeutically effective amount of testosterone by needle-free injection results in greater dispersion than administration with a needle injection.

42. The method of claim 1, wherein administering to the subject a therapeutically effective amount of testosterone by needle-free injection does not cause damage to skin cells at the site of injection.

43. A method of increasing testosterone levels in a subject in need thereof, the method comprising administering to the subject a pharmaceutical composition comprising a therapeutically effective amount of testosterone and a pharmaceutically acceptable carrier by needle-free injection.

44. A method of delivering testosterone to a subject in need thereof, the method comprising administering to the subject a pharmaceutical composition comprising a therapeutically effective amount of testosterone and a pharmaceutically acceptable carrier by needle-free injection.

45. A method of treating hypogonadism or the symptoms thereof, the method comprising administering to a subject in need thereof a therapeutically effective amount of testosterone by a needle-free injection.

46. The method of claim 45, wherein the subject is refractory to testosterone therapy.

47. A method of treating secondary hypogonadism or the symptoms thereof in a subject in need thereof, the method comprising administering to the subject a therapeutically effective amount of testosterone by a needle-free injection.

48. The method of claim 47, wherein the subject is refractory to testosterone therapy.

49. A method of administering a hormone to a subject in need thereof, the method comprising administering the hormone via a needle-free injection to an injection site with a needle-free injection device.

50. The method of claim 49, wherein the needle-free injection device comprises a portable injector and a disposable syringe.

51. The method of claim 50, wherein the disposable syringe is pre-loaded with a therapeutically effective amount of the hormone.

52. The method of claim 50, wherein the hormone is testosterone.

53. The method of claim 50, wherein the disposable syringe can be loaded with a variable amount of the hormone.

54. A method of minimizing fluctuations in testosterone levels in a subject diagnosed with hypogonadism, the method comprising:
serially administering to the subject a therapeutically effective amount of testosterone by needle-free injection to an injection site;
wherein the subjects testosterone levels are maintained within a range from between about 300 ng/dL and 900 ng/dL.

55. The method of claim 54, wherein serially administering to the subject a therapeutically effective amount of testosterone comprises at least on injection every 168 hours.

56. The method of claim 54, serially administering to the subject a therapeutically effective amount of testosterone comprises at least one injection every 24 hours.

57. The method of claim 54, serially administering to the subject a therapeutically effective amount of testosterone comprises at least one injection every 48 hours.

58. The method of claim 54, wherein administering to the subject a therapeutically effective amount of testosterone by needle-free injection is performed once every 48 hours with a 72 hour gap after three sequential administrations.
